(19) United States
(12) Patent Application Publication
(10) Pub. No.: US 2017/0252042 A1
(43) Pub. Date: Sep. 7, 2017

(54) MULTI-COMPONENT DETACHABLE CUTTING AND CLAMPING TOOL AND METHODS OF USING SAME

(71) Applicant: Novate Medical Technologies, LLC, New Orleans, LA (US)

(72) Inventors: William Christopher KETHMAN, Palo Alto, CA (US); Colin Alexander DAVIS, New Orleans, LA (US); Charles Roy DeARK, III, Mandeville, LA (US); William Richardson JONES, III, San Francisco, CA (US); Todd Elliot LEWIS, Corvallis, OR (US)

(73) Assignee: Novate Medical Technologies, LLC, New Orleans, LA (US)

(21) Appl. No.: 15/598,542

(22) Filed: May 18, 2017

Related U.S. Application Data
(63) Continuation of application No. PCT/US2015/061821, filed on Nov. 20, 2015.
(60) Provisional application No. 62/082,723, filed on Nov. 21, 2014.

(51) Int. Cl. A61B 17/122

(52) U.S. Cl. CPC A61B 17/122; A61B 2017/1225

(57) ABSTRACT

A clamping and cutting device that has a blade and blade receptacle aspect of the cutting mechanism with open clamping mechanisms that are actuated in a step-wise fashion due to a mechanical timing mechanism.

### CROSS-REFERENCE TO RELATED APPLICATION

This application is a continuation of International Application No. PCT/US2015/061821, filed Nov. 20, 2015, entitled “Multi-Component Detachable Cutting and Clamping Tool and Methods of Using Same,” which claims priority under 35 USC §119(e) and to U.S. Provisional Patent Application Ser. No. 62/082,723, filed Nov. 21, 2014, entitled “Multi-Component Detachable Cutting and Clamping Tool and Methods of Using and Making Same,” the disclosures of which are incorporated herein in their entirety.

### BACKGROUND

Field of the Invention

Disclosed embodiments relate to a multi-component detachable cutting and clamping tool in the technical field of medical devices, and more particularly in the technical field of obstetric medical devices.

Background

Maternal and infant infections represent one of the most common complications of childbirth in developing countries where most infants are born worldwide. In developing nations where health care infrastructure is commonly limited, most deliveries occur outside a hospital setting with help from family or birth attendants, resulting in up to half of all mothers and newborns not receiving skilled care during and immediately after birth. Nearly all (99%) of newborn deaths occur in low- to middle-income countries, where most birth practices involve severing the umbilical cord using non-sterile or incompletely sterile instruments that can lead to infection. It is believed that effective prenatal and postnatal care, including treatment of maternal infections during pregnancy, ensuring a clean birth, care of umbilical cord, and immediate breastfeeding could reduce up to 75% of infant deaths occurring under one-month. For these reasons, cost-effective, non-reusable, safe, and easy-to-use clamping and cutting implements are necessary components of helping reduce neonatal mortality in developing countries. In addition, tracking infant and maternal outcomes both in and outside of hospital settings in some developing countries can be difficult and so unique identifiers or radiofrequency identification (RFID) tags embedded within novel umbilical cord clamping and cutting devices or other surgical instruments could be used to improve outcome tracking. These unique identifiers could be combined with low-cost SMS-based or internet-enabled repositories or databases so that detailed analysis can be performed by aid organizations or governments so that appropriate interventions or policies may be utilized.

In developed countries, umbilical cord infections are relatively uncommon, however, additional challenges remain. The current limitations of the procedure in developed countries include: procedure duration, infant security/identification, cost, number of instruments required for both placement and sometimes removal of clamps prior to discharge from the hospital, usability, and difficulty in collecting umbilical cord blood once permanent clamps are applied. The increased duration and cost of the procedure are due to the number of reusable and disposable implements required and the need to coordinate movements accurately in a critical point in the birthing process, especially for high-risk infants and mothers. Infant security and identification is necessary in preventing infants being mistaken, lost, or stolen. While this is an uncommon occurrence, it is considered a never-event with significant resources allocated for prevention. Infant security and identification is most commonly addressed through obvious devices or bands applied either to the infant's wrist or on the clamping implement. The obvious nature of these security devices represents a fundamental weakness in infant protection. Beyond the use of unique identifiers or embedded RFID tags, local identifiers, for example, a logo of the birthing facility or color-based system, could be used to allow emergency personnel to better care for abandoned infants.

The first few weeks of a newborn mammal's life are critical to its long-term survival and health. Umbilical cord care is just as important in veterinary use as it is in human use, especially since the environment an animal is born into is usually less sterile than that of a human birth. The umbilical cord is usually severed immediately after a birth, and typically a disinfectant is applied to prevent pathogens from entering an animal's body through the cord. Umbilical cord devices can be used in veterinary medicine as an effective way to prevent umbilical cord bleeding and infection in the first few days of life. Large animal births, in particular, could benefit from an improved means of severing the umbilical cord, as many large animal births are not attended by a trained veterinarian but by a livestock handler.

A key disadvantage of the present umbilical cord clamping and cutting method in both developed and developing markets is its multi-step nature. The procedure of severing the umbilical cord in developed countries involves multiple pieces of equipment: hemostats, plastic clamps, and a cutting implement. In practice, two metal hemostats are secured to the umbilical cord in a spaced relation to one another, and the cord is then cut between the two clamps using scissors. Due to the material properties of the umbilical cord and its slippery nature after birth, this method often requires two hands and multiple attempts to sever. The metal hemostat on the baby's side is then replaced with a permanent plastic clamp and the other is removed when the placenta is discarded. Prior to leaving the hospital facility, the permanent plastic clamp is usually removed, requiring an additional hinge cutting device. In developing countries, the method for severing the umbilical cord often involves the use of clamping implements or devices, usually a type of string or plastic clamp, to stop the flow of blood and a sharp blade, often contaminated or reused, to sever the cord between the two clamped areas. There are clear opportunities for innovation in these areas.

Various surgical instruments and devices have been developed to separate and clamp the umbilical cord joining a newborn infant and the mother, however, most are reusable, difficult to use, and/or are not cost-effective. Reusability is an understandable health concern due to the possibility of contamination and subsequent infection; those that claim not to be reusable still utilize a metallic cutting implement that can be removed and reused. The metallic blade is problematic for several reasons: it is a safety hazard, can corrode prior to use, there is a risk of the blade being removed for reuse, and it is less economical to manufacture and distribute. In addition, variations on obstetric scissors do not provide adequate protection from unintended blood splatter, placing the infant, mother, and birth attendant at risk from contracting blood-borne pathogens, including HIV and Hepatitis viral infections. Furthermore, it is usually necessary to first orient the device so that the clamp side with the blade remains on the mother's side of the cord to be discarded with the placenta. Improper orientation or use of such devices is more likely to occur in developing nations due to difficulties in training and language barriers.

The phrase “elongate biological organ” as used herein is intended to connote an umbilical cord, an artery, a vein, a capillary, a conduit, a tube, a duct, and in general any flexible and/or deformable member which is capable of being clamped and then severed. Although the present invention will be referred to hereinafter in connection with the clamping and severing of an umbilical cord, the invention is not to be taken limited solely to use in connection with umbilical cords.

While certain novel features of this embodiment are shown and described below, it is not intended to be limited to the details specified, since a person of ordinary skill in the relevant art will understand that various omissions, modifications, substitutions and changes in the forms and details illustrated and in its operation may be made without departing in any way from the spirit of the embodiment. No feature is critical or essential unless it is expressly stated as being “critical” or “essential.”

### SUMMARY

This application discloses a cutter clamp assembly that can cut and clamp an elongate biological organ, such as an umbilical cord, having a cutter assembly and one or more clamps.

### BRIEF DESCRIPTION OF THE DRAWINGS

FIGS. 1A-C are schematic illustrations of a cutter clamp assembly according to an embodiment.

FIGS. 2A-C are schematic illustrations of the cutter assembly of the cutter clamp assembly of FIGS. 1A-C in open, partially closed, and fully closed configurations, respectively.

FIGS. 3A-B are schematic illustrations of a clamp of the cutter clamp assembly of FIGS. 1A-C in open and closed configurations, respectively.

FIG. 4 is a schematic illustration of the cutter clamp assembly of FIGS. 1A-C with a clamp in the closed configuration and the cutter assembly in the partially closed configuration, and an elongate biological organ disposed in the cutter clamp assembly.

FIG. 5 is a flow chart illustrating a method of cutting and clamping an elongate biological organ, according to an embodiment.

FIGS. 6A-B are perspective views of a cutter clamp assembly according to another embodiment, in an open configuration.

FIG. 6C is a perspective view of the cutter clamp assembly of FIG. 6A-B in a fully closed configuration.

FIGS. 7A-B are perspective views of the cutter assembly of the cutter clamp assembly of FIGS. 6A-C in open and fully closed configurations, respectively.

FIGS. 8A-B are perspective views of the blade portion and blade receptacle portion, respectively, of the cutter assembly of FIGS. 7A-B.

FIGS. 9A-C are perspective views of a clamp of the cutter clamp assembly of FIGS. 6A-C in an open configuration.

FIG. 9D is a perspective view of the claim of FIGS. 9A-C in a closed configuration.

FIGS. 10A-B are perspective views of the cutter clamp assembly of FIGS. 6A-C disposed about an elongate biological organ and after cutting and clamping the organ, respectively.

FIG. 10C is a perspective view of a claim of the cutter clamp assembly of FIGS. 10A-B, clamped to the cut end of the organ.

FIG. 11 is perspective view of a cutter clamp assembly having a ratchet, according to an embodiment, in an open configuration.

FIG. 12 is a perspective view of a cutter clamp assembly having a ratchet and excluding a cutter assembly latch, according to an embodiment, in an open configuration.

### DETAILED DESCRIPTION

Apparatus and methods for performing a procedure to cut and clamp an elongate biological organ, such as an umbilical cord, are described herein. In some embodiments, a method for cutting and clamping an elongate biological organ includes disposing about an elongate biological organ (e.g., an umbilical cord) a cutter clamp assembly. The cutter clamp assembly has a clamp with a lower jaw portion and an upper jaw portion coupled for relative movement between an open configuration defining an organ receiving zone therebetween and a closed configuration defining a clamped zone therebetween. The cutter clamp assembly includes a cutter assembly having a blade portion and a blade receptacle portion coupled for relative movement between a first, open configuration, a second, partially closed configuration, and a third, fully closed configuration. The blade portion includes a blade projecting toward the receptacle portion.

The cutter assembly defines a cutting zone between the blade portion and the blade receptacle portion. The blade is at least partially clear of the cutting zone when the cutter assembly is disposed in the partially closed configuration and the blade is disposed across the cutting zone when the cutter assembly is disposed in the fully closed configuration. The cutter assembly is releasably coupled to the clamp with the clamped zone of the clamp disposed laterally adjacent to the cutting zone of the cutter assembly.

The cutter clamp assembly further includes a cutting timer mechanism configured such that relative movement of the blade portion and the blade receptacle portion between the open configuration and the partially closed configuration of the cutter assembly produces relative movement of the upper jaw portion and the lower jaw portion between the open configuration and the closed configuration of the clamp. The cutting timer mechanism is configured to permit further relative movement of the blade portion and the blade receptacle portion between the partially closed configuration and the fully closed configuration of the cutter assembly without further relative movement of the upper jaw portion and the lower jaw portion of the clamp.

With the cutter clamp assembly disposed about the elongate biological organ, or more specifically, with a first portion of the organ disposed in the organ receiving zone of the clamp, and a second portion of the organ disposed in the cutting zone of the cutter assembly, manual pressure can be applied to the blade portion and the blade receptacle portion of the cutter assembly to urge the cutter assembly from the open configuration to the partially closed configuration and thus to urge the clamp from the open configuration to the closed configuration and to clamp the first portion of the elongate biological organ.

Further manual pressure can be applied to the blade portion and the blade receptacle portion to urge the cutter assembly from the partially closed configuration to the fully closed configuration and to cut the second portion of the organ. Upon cutting of the second portion of the organ, the clamp can be separated from the cutter assembly, with the clamp at least temporarily retained on the second portion of the organ.

In some embodiments, a cutter clamp assembly is provided to perform the above cutting and clamping procedure. Such an assembly can include, for example, a clamp, a cutter assembly, and a cutting timer mechanism. The clamp can include a lower jaw portion and an upper jaw portion coupled for relative movement between a first, open configuration and a second, closed configuration. The clamp can define between the upper jaw portion and the lower jaw portion an organ receiving zone in the open configuration of the clamp and a clamped zone between the upper jaw portion and the lower jaw portion in the closed configuration of the clamp. The clamp can be configured to receive a first portion of an elongate compressible biological organ in the organ receiving zone and to compress the received first portion of the organ between the upper jaw portion and the lower jaw portion into the clamped zone.

The cutter assembly of the cutter clamp assembly can include a first portion and a second portion coupled for relative movement between a first, open configuration, a second, partially closed configuration, and a third, fully closed configuration. Either the first portion or the second portion can include a blade projecting toward the other of the first portion and the second portion. The cutter assembly can further define a cutting zone between the first portion and the second portion such that the blade is at least partially clear of the cutting zone when the cutter assembly is disposed in the partially closed configuration, and the blade is disposed completely across the cutting zone when the cutter assembly is disposed in the fully closed configuration.

The cutter assembly can be releasably coupled to the clamp by a mounting connection between the lower jaw portion of the clamp and the second portion of the cutter assembly, with the clamped zone of the clamp disposed laterally adjacent to the cutting zone of the cutter assembly, such that a second portion of the elongate biological organ can be received in the cutting zone when the first portion of the organ is disposed in the clamped zone.

The cutting timer mechanism of the cutter clamp assembly can include a first timer portion disposed on the upper jaw portion of the clamp and a second timer portion disposed on the first portion of the cutter assembly. The first timer portion can be engageable with the second timer portion such that relative movement of the first portion and the second portion of the cutter assembly between the open configuration and the partially closed configuration of the cutter assembly produces relative movement of the upper jaw portion and the lower jaw portion between the open configuration and the closed configuration of the clamp. The cutting timer mechanism can be configured to permit further relative movement of the first portion and the second portion of the cutter assembly between the partially closed configuration and the fully closed configuration of the cutter assembly without further relative movement of the upper jaw portion and the lower jaw portion of the clamp.

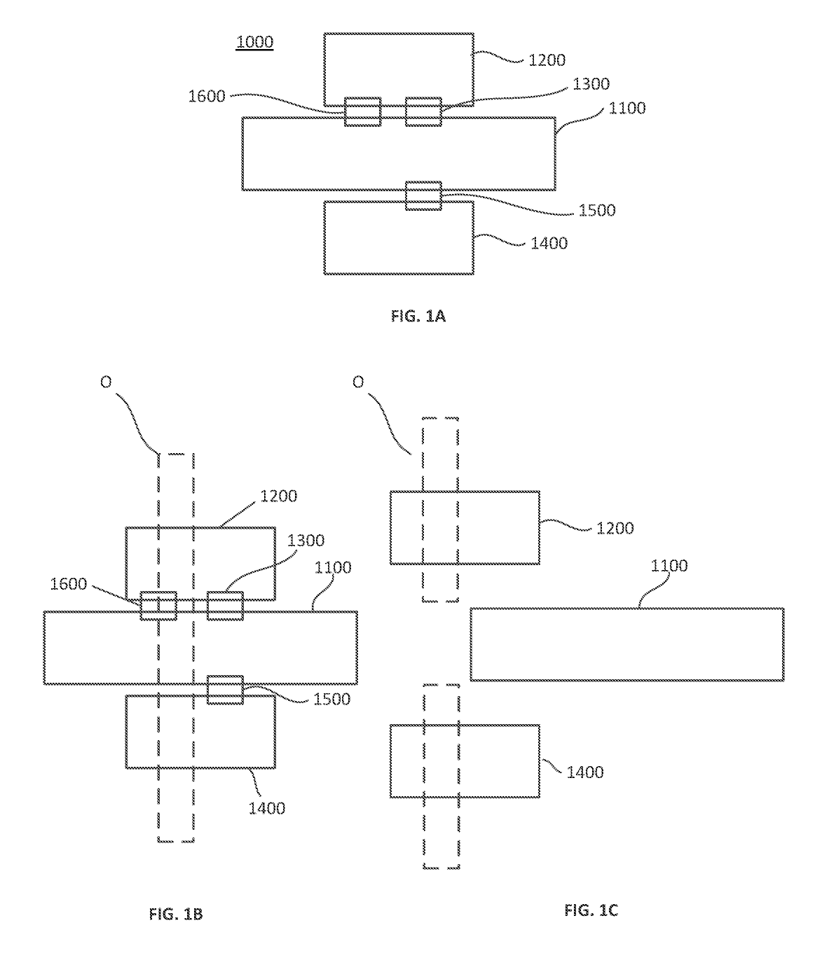

FIG. 1A is a schematic illustration of a cutter clamp assembly, according to an embodiment. The cutter clamp assembly 1000 can include a cutter assembly 1100 configured to cut an elongate biological organ, a first clamp 1200 configured to clamp a portion of the organ and removably coupled to the cutter assembly 1100 via a first clamp mount 1300, a second clamp 1400 configured to clamp a portion of the organ and removably coupled to the cutter assembly 1100 via a second clamp mount 1500, and a cutting timer mechanism 1600 coupled to the cutter assembly 1100 and the first clamp 1200.

The cutter clamp assembly 1000 can be disposed about an elongate biological organ O (also referred to herein as “organ”), as shown in FIG. 1B. More specifically, the organ O can be placed in a desired position relative to the cutter clamp assembly 1000 in which a first portion of the organ O is disposed in a cutting zone of the cutter assembly 1100, a second portion of the organ O is disposed in an organ receiving zone of the first clamp 1200, and a third portion of the organ O is disposed in an organ receiving zone of the second clamp 1400, the first portion of the organ O being disposed between both the second and third portions of the organ O. When the organ O is in the desired position relative to the cutter clamp assembly 1000, the cutter clamp assembly 1000 can be actuated to transition (1) the first clamp 1200 from its open configuration to its closed configuration and to clamp the second portion of the organ O, and (2) the second clamp 1400 from its open configuration to its closed configuration and to clamp the third portion of the organ O.

The cutter clamp assembly 1000 can be further actuated to transition the cutter assembly 1100 from its open configuration to its closed configuration and to cut the first portion of the organ. In some embodiments, when the cutter clamp assembly 1000 is actuated to transition the first clamp 1200 to its closed configuration and the second clamp 1400 to its closed configuration, the cutter assembly 1100 is also transitioned. In such embodiments, upon actuation of the cutter clamp assembly 1000, the cutter assembly 1100 is transitioned from its open configuration to a partially closed configuration. Upon proper clamping of the organ O between the first clamp 1200 and the second clamp 1400, the cutter assembly 1000 can be further actuated to transition the cutter assembly 1100 from its partially closed configuration to a fully closed configuration and to cut the first portion of the organ. In this manner, the cutter clamp assembly 1000 can both clamp and cut an organ O in response to a continuous force or movement applied to the cutter clamp assembly 1000, e.g., a continuous manual movement provided by an operator of the cutter clamp assembly 1000. Enabling both cutting and clamping in response to a continuous manual movement can simplify the procedure and promote repeatable safe and effective operations.

As shown in FIG. 1C, after the cutter clamp assembly 1000 has clamped and cut the organ O, the first clamp 1200 and the second clamp 1400 can be decoupled or otherwise separated from the cutter assembly 1100, with the first clamp 1200 removably coupled to or retained on the second portion of the organ O and the second clamp 1400 removably coupled to or retained on the third portion of the organ O. For example, during a procedure on an umbilical cord, after the cutter clamp assembly 1000 has clamped and cut the umbilical cord, the first clamp 1200 and the second clamp 1400 can be separated from the cutter assembly 1100, with the first clamp 1200 retained on a portion of the umbilical cord that is attached to a placenta, and the second clamp 1400 retained on a portion of the umbilical cord that is attached to a newborn.

Upon separation of both the first clamp 1200 and the second clamp 1400 from the cutter assembly 1100, one or both of the first clamp 1200 and the second clamp 1400 can be independently transitioned from its closed configuration to its opened position to separate one or both of the first clamp 1200 and the second clamp 1400 from the organ O, to sample contents of the organ O, or to readjust one or both of the first clamp 1200 and the second clamp's 1400 position relative to the organ O and then independently re-transition one or both of the first clamp 1200 and the second clamp 1400 from its opened configuration to its closed configuration to re-clamp the organ O.

Although the cutter clamp assembly 1000 has been shown and described as having two clamps (i.e., the first clamp 1200 and the second clamp 1400), in other embodiments, the cutter clamp assembly 1000 can have any suitable number of clamps configured to clamp a portion or portions of an elongate biological organ. For example, in alternative embodiments, a cutter clamp assembly can include only one clamp configured to clamp a portion of an elongate biological organ.

In some embodiments, the clamp(s) (e.g., the first clamp 1200 and/or the second clamp 1400) can include one or more indicia of identification. Indicia of identification, for example, can include a unique identification code or symbol, radio-frequency identification (RFID), a bar code, a logo, a color code, etc. The indicia of identification can be associated with, for example, a particular health care facility, a particular patient or relative of the patient, a particular geographical region, etc. In some instances, for example in developed countries, the indicia of identification can help identify abandoned newborns, and in developing countries, the indicia of identification can aid in the collection of regional and national health outcome statistics. In some embodiments, alternatively or in addition to the indicia of identification on the one or more clamps, indicia of identification can be disposed on the cutter assembly.

In some embodiments, the clamps (e.g., the first clamp 1200 and the second clamp 1400) can include matching or corresponding identification or visual indicium. For example, after an umbilical cord procedure (i.e., after clamping and cutting of the umbilical cord), the clamp attached to the placenta side of the cord can be matched to the clamp attached to the newborn side of the cord. In this manner, a health care practitioner, for example, can identify or match the placenta to the corresponding newborn from which it was previously attached.

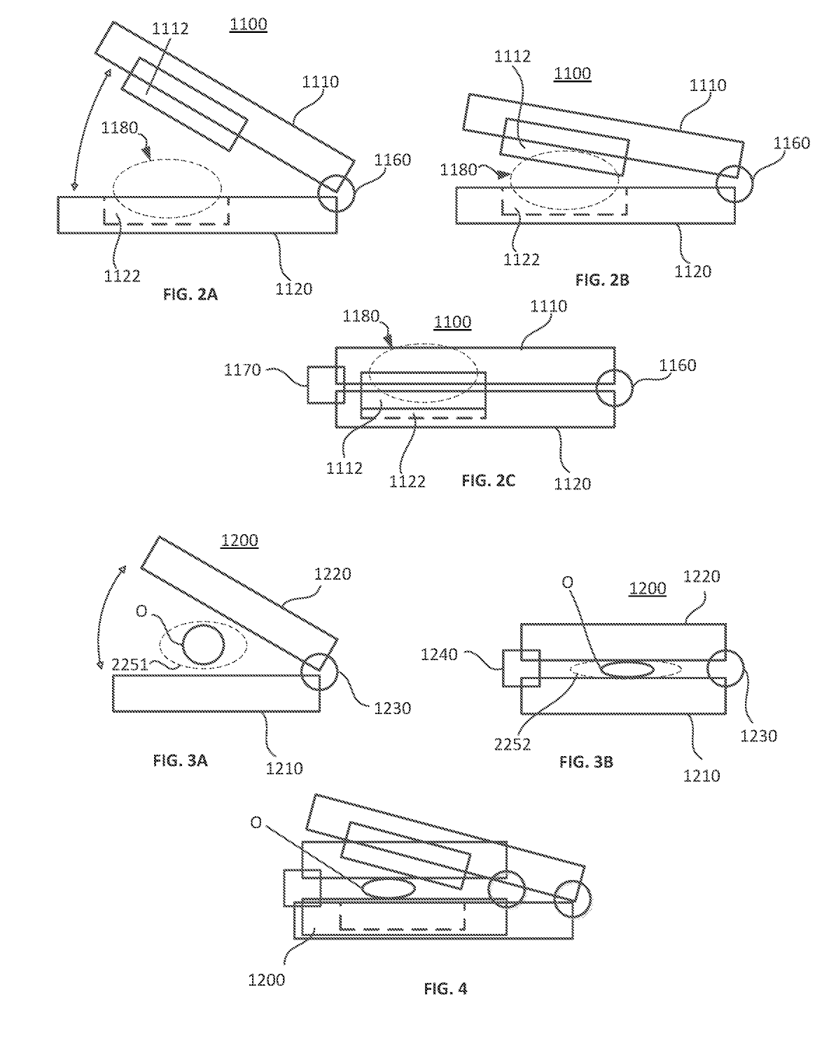

FIGS. 2A-C are schematic illustrations of the cutter assembly 1100 of the cutter clamp assembly 1000 of FIGS. 1A-C in open, partially closed, and fully closed configurations, respectively. As shown, the cutter assembly 1100 includes a blade receptacle portion 1120 and a blade portion 1110 rotationally coupled or hinged to the blade receptacle portion 1120 via a cutter assembly hinge 1160. The cutter assembly hinge 1160 is configured to promote relative angular movement or otherwise provide a pivot point between the blade portion 1110 and the receptacle portion 1120. In this manner, as discussed above with respect to FIGS. 1A-1C, transition of the cutter assembly 1100 between configurations (e.g., open configuration, partially closed configuration, fully closed configuration) can include the blade portion 1110 and the blade receptacle portion 1120 rotating relative to each other about the cutter assembly hinge 1160. A cutting zone 1180 is defined between the blade portion 1110 and the blade receptacle portion 1122. As shown, the cutting zone 1180 is defined at least partially within the blade receptacle 1122.

The blade portion 1110 includes a blade 1112 configured to pierce or cut the organ O when placed into contact with the organ O. As shown, the blade 1112 projects toward the blade receptacle portion 1122. As shown across FIGS. 2A-2C, when the cutter assembly 1100 is in its open configuration (FIG. 2A), the blade 1112 is clear of the cutting zone 1180. When the cutter assembly 1100 is in its partially closed configuration (FIG. 2B), the blade 1112 is at least partially clear of the cutting zone 1180. When the cutter assembly 1100 in in its fully closed configuration (FIG. 2C), the blade 1112 is disposed completely across the cutting zone. In such embodiments, in use, in some instances, the blade 1112 may make contact with and at least partially pierce (e.g., prior to severance of the organ O) the organ O when the cutter assembly 1100 is in its partially closed configuration. In alternative embodiments, unlike as illustrated in FIG. 2B, the blade may be completely clear of the cutting zone when the cutter assembly is disposed in its partially closed configuration.

The blade 1112 can be shaped and sized in any suitable manner configured to cut the organ O. For example, in some embodiments, the blade 1112 can be constructed at least partially from non-metallic materials to promote safety for users and patients of the cutter clamp assembly 1000. As such, the blade 1112 can be configured to cut an elongate biological organ while limiting the blade's 1112 ability to undesirably cut, for example, a finger of a user of the cutter clamp assembly 1000. Further, the non-metallic construction of the blade 1112 can limit or prevent reuse or multiple reuses of the cutter clamp assembly 1000, thereby limiting or reducing the likelihood of insanitary uses of the cutter clamp assembly 1000. Even further, the non-metallic construction of the blade 1112 can promote a more cost-effective cutter clamp assembly 1000, due in part to the cost-effective manufacturing processes (e.g., injection molding) available in connection with such materials. As a further example, in some embodiments, the blade 1112 can be constructed from degradable materials (e.g., a degradable polymer) to reduce waste and waste collection, particularly for example, in developing countries or regions without suitable and safe waste collection. As a further example, in some embodiments, the blade 1112 can be constructed with anti-microbial additive materials (e.g., an antimicrobial composite polymer) to discourage colonization of a bacterial, for example, in developing countries or regions. The blade 1112 and the blade portion 1110 can be monolithically formed, or alternatively, the blade 1112 and the blade portion 1110 can be formed separately and then joined together.

The blade receptacle portion 1120 defines a blade receptacle 1122 configured to receive a portion of the blade 1112. For example, when the cutter assembly 1100 is in its fully closed configuration (FIG. 2C), a portion of the blade 1112 is disposed across or within the blade receptacle 1122. In this manner, in use, the blade 1112 can sever and pass through a portion of the organ as the blade 1112 transitions from being clear of the cutter zone 1180 to at least partially being disposed within the receptacle 1122.

To cut an elongate biological organ (e.g., an umbilical cord), the cutter assembly 1100 can be manipulated from a first, open configuration, to a second, partially closed configuration, to a third, fully closed configuration, as shown in sequence in FIGS. 2A-2C, respectively. With the cutter assembly 1100 disposed about the organ (i.e., between the blade portion 1110 and the blade receptacle portion 1120, the cutter assembly 1100 can be manipulated (e.g., by a manual force applied by a user to the blade portion 1110 and/or the blade receptacle portion 1120) to transition the cutter assembly 1100 from its open configuration (FIG. 2A) to its partially closed configuration (FIG. 2B). Although not shown in FIG. 2B, as described above with reference to FIGS. 1A-1C, and described further herein with respect to FIGS. 3A, 3B and 4, the first clamp 1200 and the second clamp 1400 are configured to clamp portions of the organ when the cutter assembly 1100 is in its partially closed configuration (FIG. 2B) and coupled to the first clamp 1200 and the second clamp 1400.

As shown in sequence from FIGS. 2B to 2C, the cutter assembly 1100 can be further manipulated (e.g., by the manual force) to transition the cutter assembly 1100 from its partially closed configuration (FIG. 2B) to its fully closed configuration (FIG. 2C) (i.e., the blade portion 1110 and the blade receptacle portion pivot about the cutter assembly hinge 1160) and to cut or sever the organ (not shown). As shown in FIG. 2C, the blade 1112 of the blade portion 1110 is disposed within the blade receptacle 1122 of the blade receptacle portion 1120 when the cutter assembly 1100 is in its fully closed configuration. When the cutter assembly 1100 is in its fully closed configuration (FIG. 2C), the cutter assembly latch 1170 can be actuated to lock or limit relative movement between the blade portion 1110 and the blade receptacle 1122.

In some embodiments, the cutter assembly latch 1170 can be reversibly actuate-able such that the latch 1170 can temporarily lock the cutter assembly 1100 in its fully closed configuration (e.g., during storage prior to use and/or after use). In alternative embodiments, the cutter assembly latch 1170 can be substantially permanently actuated such that the cutter assembly 1100 is locked in its fully closed configuration when the cutter assembly latch 1170 is engaged. In such embodiments, the cutter assembly latch 1170 can limit or prevent reuse of the cutter assembly 1100 and potential contamination resulting from such reuse. Further, in yet alternative embodiments, a cutter assembly can include multiple cutter assembly latches. For example, in such embodiments, the cutter assembly can include a first cutter assembly latch to temporarily lock the cutter assembly in its fully closed configuration (e.g., for safe and sanitary storage prior to use), and a second cutter assembly latch to substantially permanently lock the cutter assembly in its fully closed configuration (e.g., for safe and sanitary storage after use, and to prevent reuse). In some embodiments, the latch 1170 can self-engage in response to the force applied to transition the cutter assembly 1100 from its partially closed configuration to its fully closed configuration. Such self-engagement (e.g., via an interference fit) can provide for desirable locking of the cutter assembly, as discussed above, without requiring a user of the assembly from separately manipulating the latch. In yet alternative embodiments, the latch 1170 can require manipulation by a user to engage.

Although the cutter assembly latch 1170 is shown and described as moving pivotally about a hinge, in alternative embodiments, a cutter assembly latch 1170 can move linearly with alternative mechanisms for connecting the moveable portions, e.g., the blade portion 2110 and the blade receptacle portion 2130.

FIGS. 3A and 3B are schematic illustrations of the first clamp 1200 of the cutter clamp assembly 1000 of FIGS. 1A-C in open and closed configurations, respectively. For efficiency, only the first clamp 1200 will be discussed, however, it should be understood that the second clamp 1400 can be the same as or similar to the first clamp 1200. As shown, the first clamp 1200 includes a first clamp upper jaw portion 1220 and a first clamp lower jaw portion 1210 rotationally coupled or hinged to the first clamp upper jaw portion 1220 via a first clamp hinge 1230.

The first clamp hinge 1230 is configured to promote relative angular movement or otherwise provide a pivot point between the first clamp upper jaw portion 1220 and the first clamp lower jaw portion 1210. In this manner, as discussed above with respect to FIGS. 1A-1C, transition of the cutter clamp assembly 1000 between configurations (e.g., from open configuration to partially closed configuration) can include the first clamp upper jaw portion 1220 and the first clamp lower jaw portion 1210 rotating relative to each other about the first clamp hinge 1230.

To clamp the organ O (e.g., an umbilical cord), the first clamp 1200 can be manipulated from a first, open configuration, to a second, closed configuration, as shown in sequence in FIGS. 3A and 3B, respectively. With the first clamp 1200 disposed about the organ O (i.e., the organ O is positioned within the first clamp organ receiving zone 1251 defined between the first clamp upper jaw portion 1220 and the first clamp lower jaw portion 1210), the first clamp 1200 can be manipulated (e.g., by a manual force applied by a user to the first clamp upper jaw portion 1220 and/or the first clamp lower jaw portion 1210) to transition the first clamp 1200 from its open configuration (FIG. 3A) to its closed configuration (FIG. 3B). As shown schematically in FIG. 3B, the organ O is clamped and deformed within the first clamp 1200 clamped zone 2252.

Further, as shown in FIG. 3B, the first clamp 1200 includes a first clamp latch 1240. When the first clamp 1200 is in its closed configuration (FIG. 3B), the first clamp latch 1240 can be actuated to lock or limit relative movement between the first clamp upper jaw portion 1220 and the first clamp lower jaw portion 1210. In this manner, the first clamp 1200 can be secured in its closed configuration during storage prior to use and/or after use. Further, after use, i.e., when a portion of an organ is clamped by the first clamp 1200, the first clamp latch 1240 can secure the organ in its clamped or deformed configuration (e.g., to promote sanitation and limit undesirable contamination of the organ or patient). The first clamp 1200 can be further manipulated to disengage the first clamp latch 1240 and/or transition the first clamp 1200 from its closed configuration to its open configuration. For example, in a procedure involving clamping an umbilical cord, the first clamp 1200 can clamp the umbilical cord to facilitate the cutting of the umbilical cord, and then the first clamp 1200 can be disengaged (moved from its closed configuration to its open configuration) or separated from the umbilical cord such that blood can be withdrawn from the umbilical cord, the first clamp 1200 can be repositioned and re-engaged, or simply removed from the cord.

In some embodiments, the latch 1240 can self-engage in response to the force applied to transition the first clamp 1200 from its open configuration to its closed configuration. Such self-engagement (e.g., via an interference fit) can provide for desirable locking of the first clamp 1200, as discussed above, without requiring a user of the cutter clamp assembly 1000 from separately manipulating the latch 1240. In alternative embodiments, the latch may require manipulation of the latch by a user to engage the latch. In alternative embodiments, a latch can be substantially permanently engaged such that the latch locks the first clamp in its closed configuration. In such embodiments, the latch can limit or prevent reuse of the clamp and potential complications resulting from removal of the latch from the organ or patient. For example, in procedures in which umbilical cord blood collection is not performed, the latch can remain engaged such that the organ remains clamped by the latch. In such cases, in some instances, the latch can remain clamped about the organ at least until the organ is detached from the patient or when a trained healthcare personnel removes the clamp.

Although the latch 1240 is shown and described as moving pivotally about a hinge, in alternative embodiments, a latch can move linearly with alternative mechanisms for connecting movable portions.

FIG. 4 is a side-view schematic illustration of the cutter clamp assembly 1000 with the first clamp 1200 in its closed configuration and clamped about the organ O, and the cutter assembly 1100 in its partially closed configuration. With the organ O clamped by the first clamp 1200, the organ O is positioned and arranged to be cut by the cutter assembly 1100. Said another way, the cutter clamp assembly 1000 is configured such that, in use, the first clamp 1200 clamps the organ O such that the organ O is secured (e.g., prior to cutting or severing of the organ O) in a position to promote proper cutting of the organ O by the cutter assembly 1100.

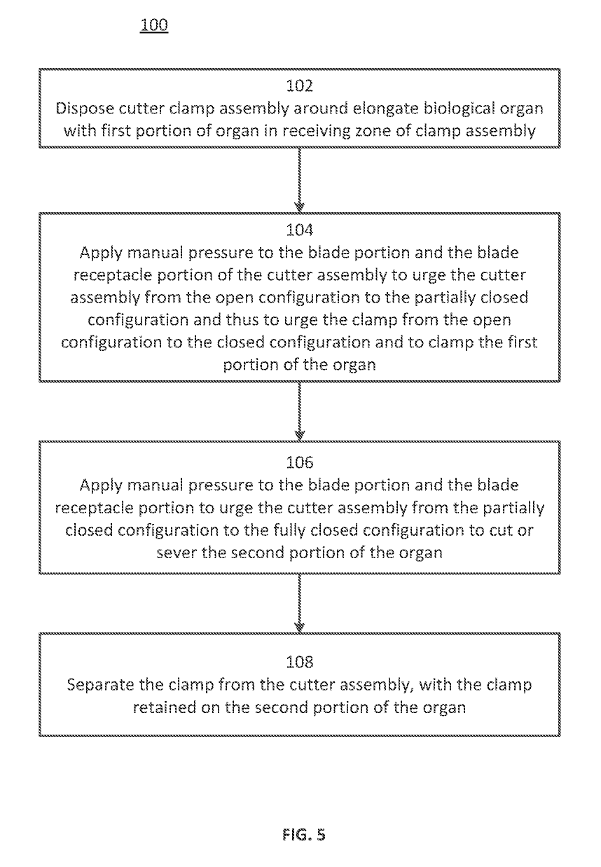

FIG. 5 shows a schematic flow diagram of a method of cutting and clamping an elongate biological organ, according to an embodiment. The method 100 includes disposing a cutter clamp assembly (e.g., the cutter clamp assembly 1000 or any other cutter clamp assembly described herein) around an elongate biological organ (e.g., an umbilical cord), at 102.

The cutter clamp assembly can include a clamp with a lower jaw portion and an upper jaw portion coupled for relative movement between an open configuration defining an organ receiving zone therebetween and a closed configuration defining a clamped zone therebetween. The cutter clamp assembly can further include a cutter assembly having a blade portion and a blade receptacle portion coupled for relative movement between a first, open configuration, a second, partially closed configuration, and a third, fully closed configuration. The blade portion can include a blade projecting toward the receptacle portion, and the cutter assembly can define a cutting zone between the blade portion and the blade receptacle portion. In such a manner, the blade is clear of the cutting zone when the cutter assembly is disposed in the partially closed configuration and the blade is disposed across the cutting zone when the cutter assembly is disposed in the fully closed configuration.

The cutter assembly can be releasably coupled to the clamp with the clamped zone of the clamp disposed laterally adjacent to the cutting zone of the cutter assembly. The cutter clamp assembly can further include a cutting timer mechanism configured such that relative movement of the blade portion and the blade receptacle portion between the open configuration and the partially closed configuration of the cutter assembly produces relative movement of the upper jaw portion and the lower jaw portion between the open configuration and the closed configuration of the clamp. The cutting timer mechanism can permit further relative movement of the blade portion and the blade receptacle portion between the partially closed configuration and the fully closed configuration of the cutter assembly without further relative movement of the upper jaw portion and the lower jaw portion of the clamp.

At 102, a first portion of the organ is disposed in the organ receiving zone of the clamp, and a second portion of the organ is disposed in the cutting zone of the cutter assembly (e.g., the cutting zone being laterally off-set from the organ receiving zone of the clamp). In this manner, a user can visually confirm proper positioning of the organ relative to the cutter clamp assembly prior to clamping or cutting of the organ, and the cutting and clamping of the organ can be temporally off-set.

The method 100 further includes applying manual pressure to the blade portion and the blade receptacle portion of the cutter assembly to urge the cutter assembly from the open configuration to the partially closed configuration and thus to urge the clamp from the open configuration to the closed configuration and to clamp the first portion of the organ, at 104. In this manner, the first portion of the organ can be clamped and secured such that the second portion of the organ is suitably arranged to be cut by the cutter assembly. For example, with the first portion of the organ being clamped when the second portion of the organ is cut, fluids within the organ are limited or prevented from undesirably flowing out of the organ through the opening caused by the cutting. Further, with the organ clamped prior to severance of the organ, potential contamination or infection of the organ or patient (e.g., newborn and/or mother) is limited.

The method 100 further includes applying further manual pressure to the blade portion and the blade receptacle portion to urge the cutter assembly from the partially closed configuration to the fully closed configuration to cut or sever the second portion of the organ, at 106.

The method 100 further includes separating the clamp from the cutter assembly, with the clamp retained on the second portion of the organ, at 108. In this manner, the second portion of the organ can remain clamped when separated from the cutter assembly.

In some embodiments, a method can further include disengaging the clamp (e.g., causing the clamp to transition from its closed configuration to its open configuration) to reposition and reclamp the organ, withdrawn organ fluid or blood, or replace with a different clamp.

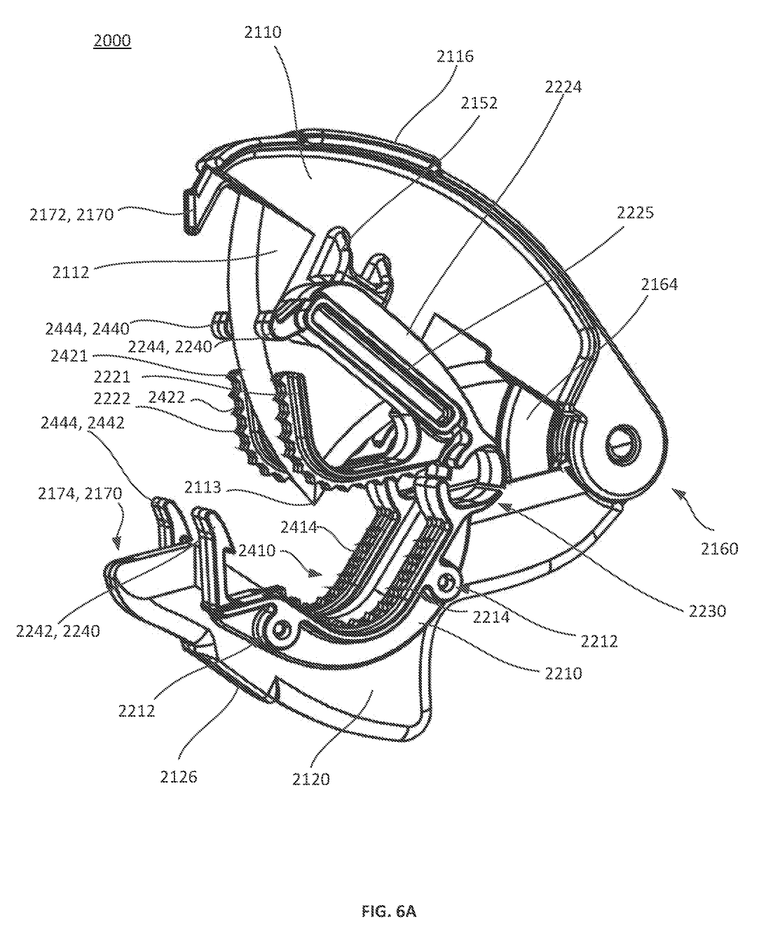

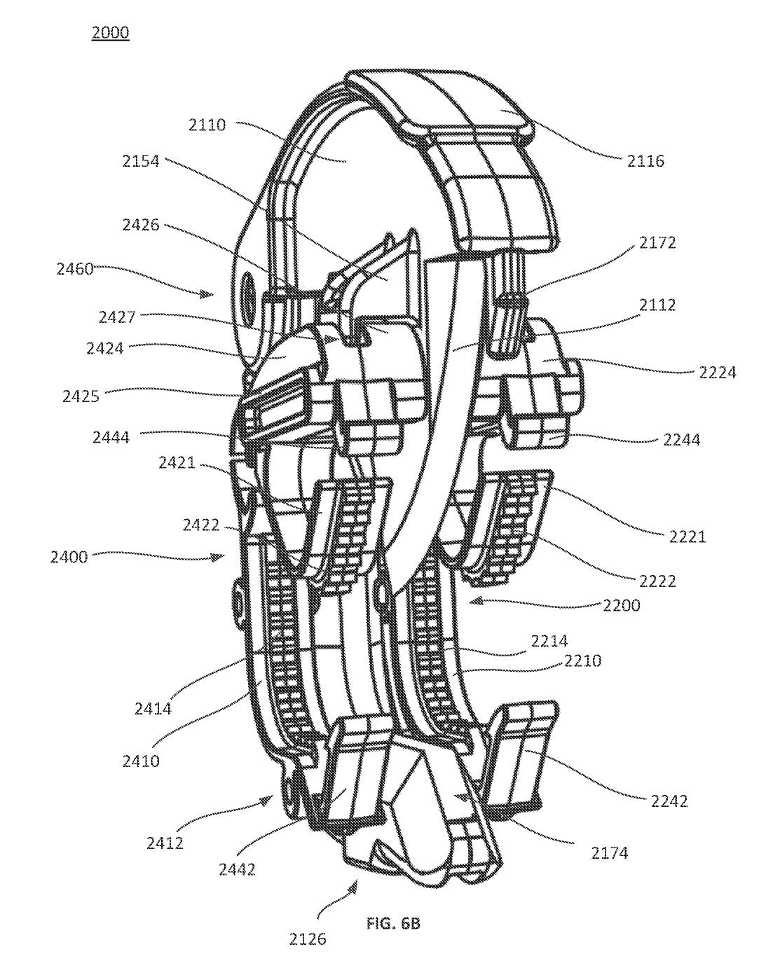

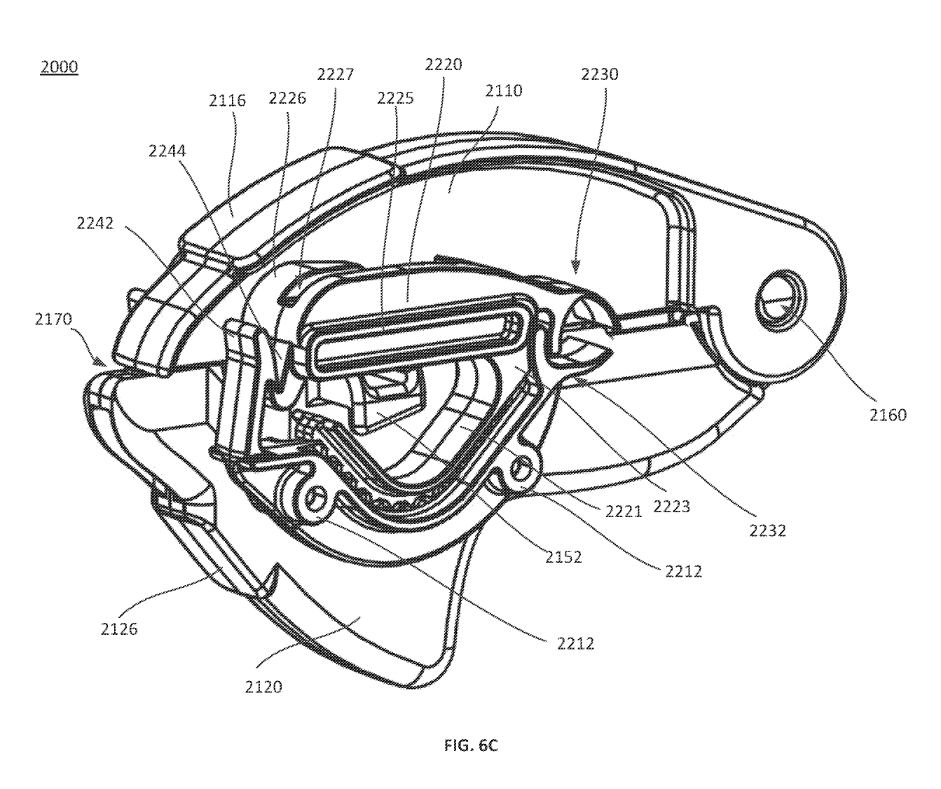

FIGS. 6A and 6B are perspective views of a cutter clamp assembly 2000 according to another embodiment, in an open configuration. FIG. 6C is a perspective view of the cutter clamp assembly 2000 in a fully closed configuration. The cutter clamp assembly 2000 includes a cutter assembly 2100 configured to cut an elongate biological organ, a first clamp 2200 configured to clamp a portion of the organ and removably coupled to the cutter assembly 2100 via a first clamp mount 2300, and a second clamp 2400 configured to clamp a portion of the organ and removably coupled to the cutter assembly 2100 via a second clamp mount 2500.

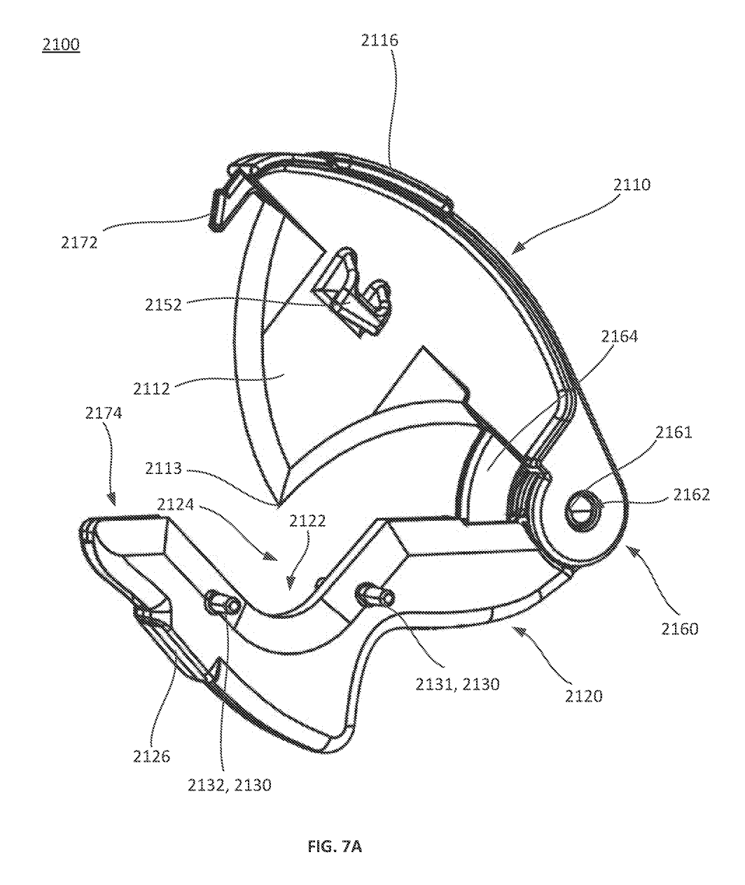

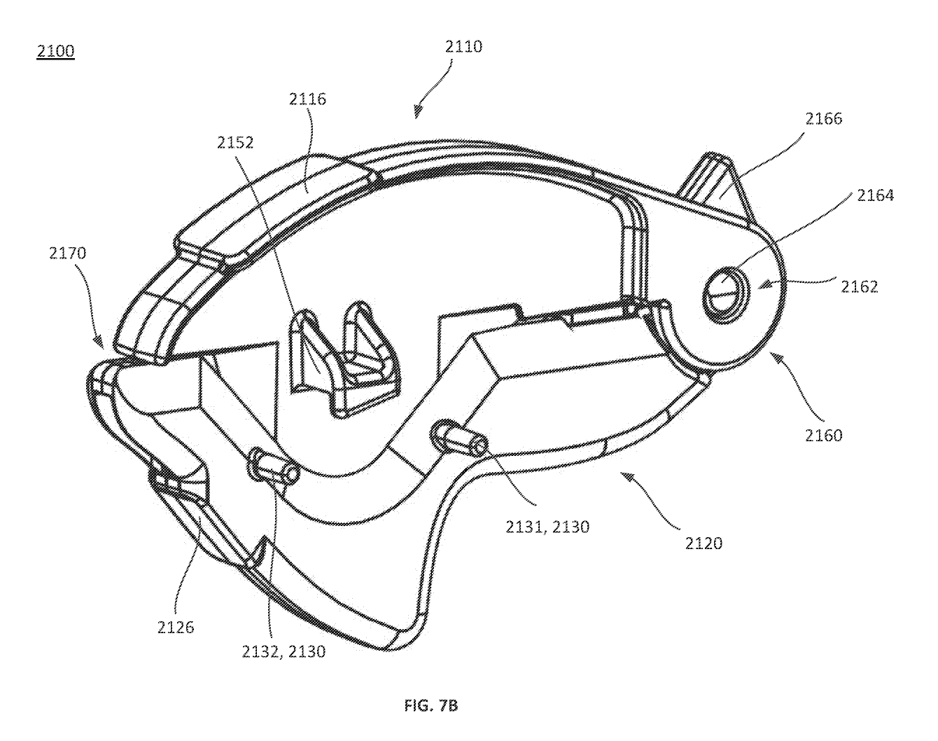

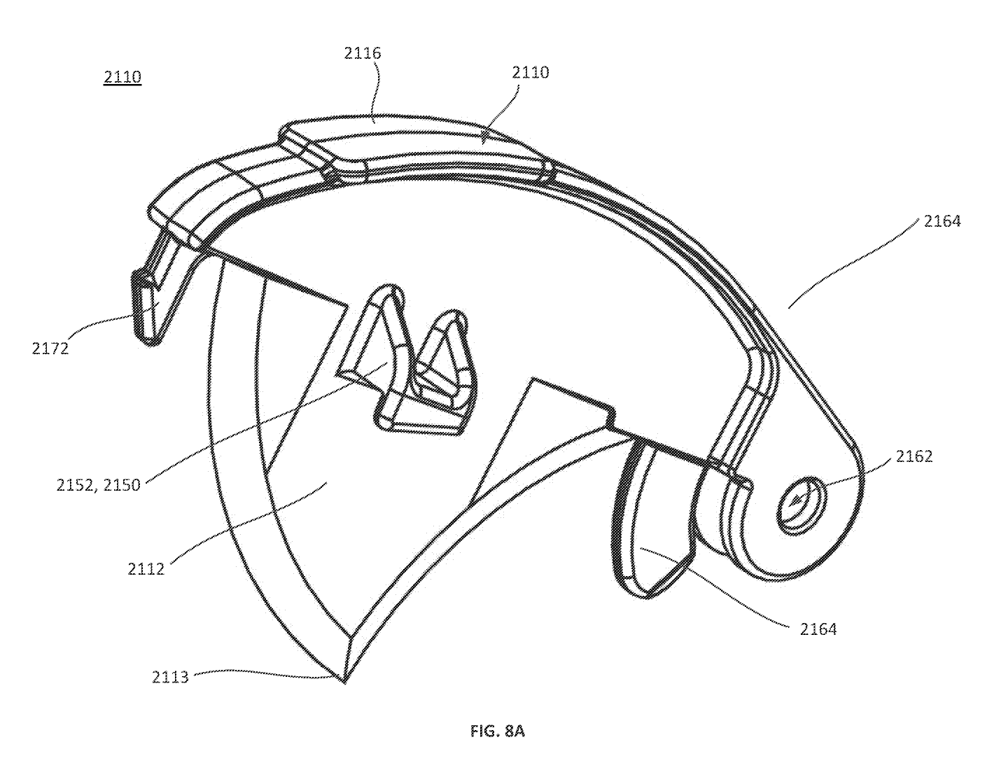

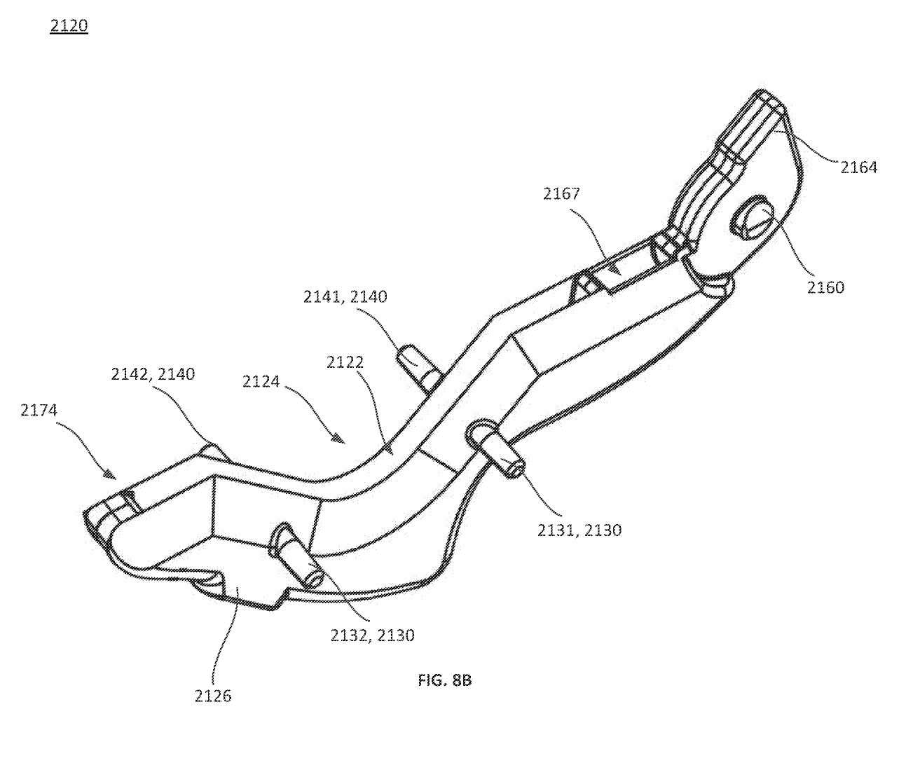

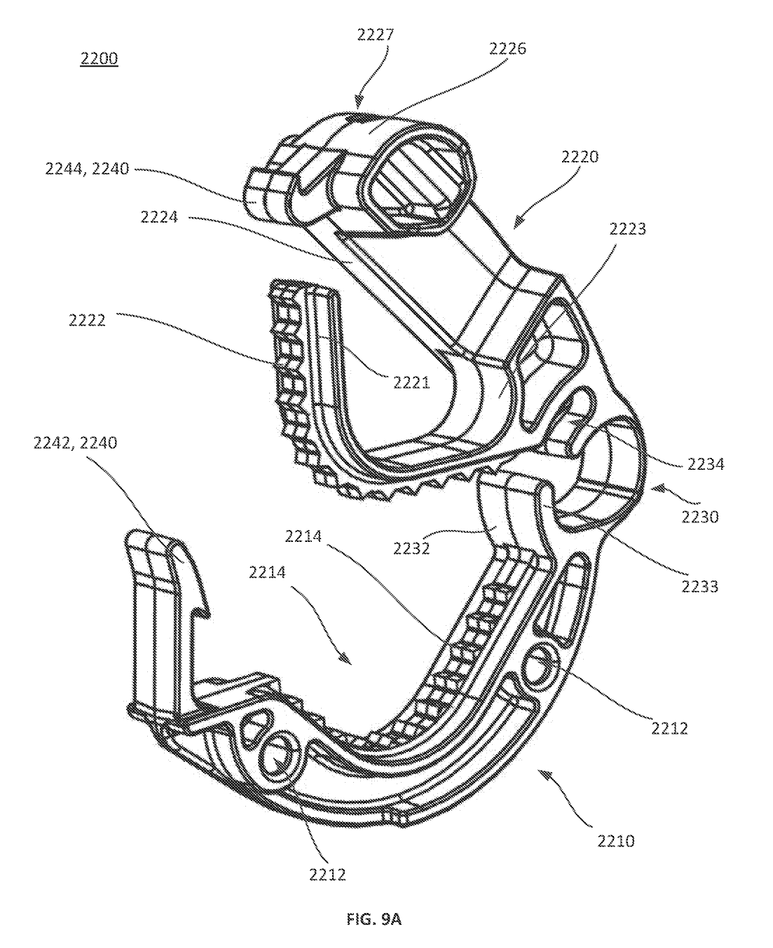

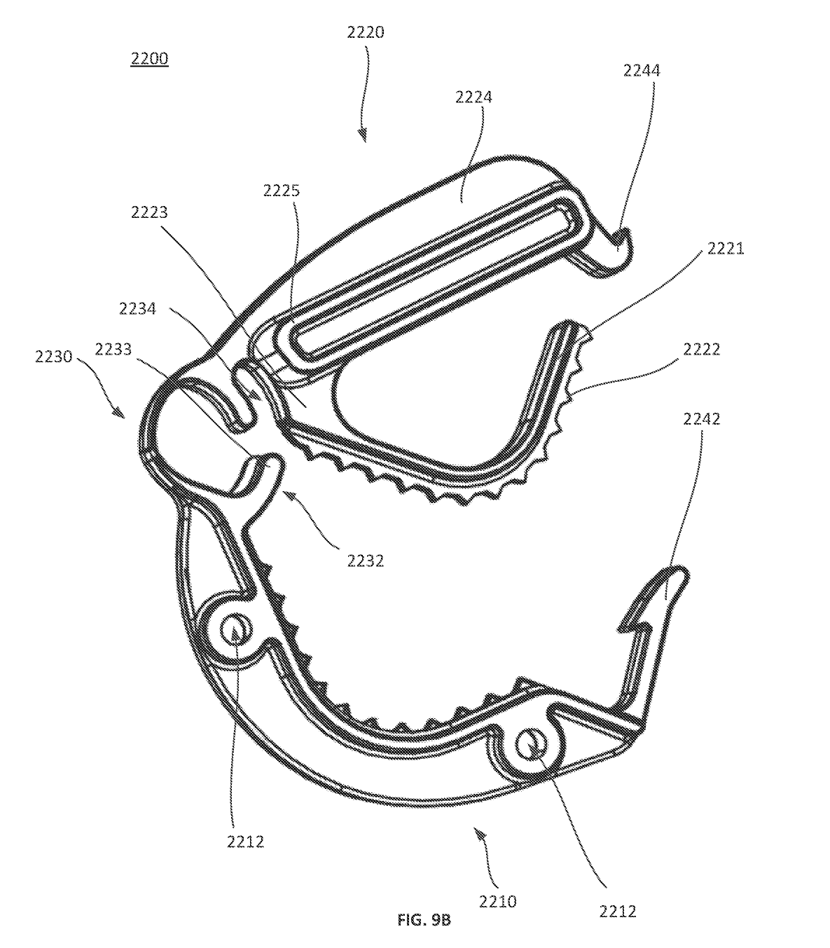

The cutter assembly 2100 of the cutter clamp assembly 2000, as illustrated in FIG. 7A in perspective view, in an open configuration, and in FIG. 7B in perspective view, in a closed configuration, includes a blade portion 2110 and a blade receptacle portion 2120 rotatably coupled to the blade portion 2110 via a cutter assembly hinge 2160. The blade portion 2110, as illustrated in FIG. 8A in perspective view, includes a blade 2112 configured to pierce, cut, and/or sever an elongate biological organ (not shown). The blade 2112 includes a blade tip 2113 configured to initiate a cut or pierce of an elongate biological organ. The blade portion 2110 further includes a blade portion grip 2116 ergonomically designed to facilitate gripping, actuating, or otherwise manipulating of the cutter clamp assembly 2000 by a user. As shown, the blade 2112 projects toward the blade receptacle portion 2122 of the cutter assembly 2100.

As shown in FIGS. 6A-C, the blade receptacle portion 2120 (illustrated in FIG. 8B in perspective view) of the cutter assembly 2100 is rotationally coupled or hinged to the blade portion 2110 via the cutter assembly hinge 2160. The cutter assembly hinge 2160 is configured to promote relative angular movement or otherwise provide a pivot point between the blade portion 2110 and the blade receptacle portion 2120. In this manner, transition of the cutter assembly 2110 between configurations (e.g., open configuration, partially closed configuration, fully closed configuration) includes the blade portion 2110 and the blade receptacle portion 2120 rotating relative to each other about the cutter assembly hinge 2160.

The blade receptacle portion 2120 includes a blade receptacle 2122 configured to receive and at least partially surround the blade 2112 when the cutter assembly 2100 is in its partially closed configuration and/or its fully closed configuration. In this manner, the blade tip 2113 can be disposed within the blade receptacle 2122, e.g., during storage of the cutter assembly 2100 or upon completion of a procedure, to limit or prevent undesirable or accidental cutting. The blade receptacle portion 2120 further includes a blade receptacle organ receiver 2124 configured to receive and/or guide a portion of the organ prior to cutting of the organ. The blade receptacle organ receiver 2124 provides repeatable and easy positioning of a portion of the organ in preparation for cutting of the organ by the blade 2112, and provides suitable seating of the portion of the organ during the cutting of the same. For example, in use, a portion of an organ can be positioned across the blade receptacle organ receiver 2124 when the cutter assembly 2100 is in its open configuration. Further, the cutter assembly 2100 can be actuated such that the blade 2112 pierces the organ when the organ is disposed within the blade receptacle organ receiver 2124. In this manner, the blade receptacle organ receiver 2124 at least temporarily contains the organ and provides a surface or counter-force to facilitate cutting or severing of the organ, and limits or prevents the organ from undesirably slipping or sliding away from the blade portion 2110 upon, e.g., upon contact with the blade 2112.

The blade receptacle portion 2120 further includes a blade receptacle portion grip 2126 ergonomically designed to facilitate gripping, actuating, or otherwise manipulating of the cutter clamp assembly 2000 by a user. In this manner, in use, a user can grip both the blade receptacle portion grip 2126 and the blade portion grip 2116 and apply forces (e.g., a user can squeeze both grips, with one or two hands) thereto to actuate the cutter clamp assembly 2100 to cut or sever an organ.

The cutter assembly hinge 2160 of the cutter assembly 2100 includes a cutter hinge pin 2161 rotatably coupled to and disposed within a cutter hinge socket 2162. The cutter assembly hinge 2160 further includes a cutter hinge stabilizer 2163 configured to stabilize or guide the cutter assembly 2100 between configurations. As shown, the cutter hinge stabilizer 2163 includes a cutter hinge stabilizer projection 2164 (projecting from the blade portion 2110) and a cutter hinge stabilizer receptacle (or guide) 2167 configured to receive the projection 2164 to provide stiffening or stabilization (e.g., limit undesirably lateral movement) between the blade portion 2110 and the blade receptacle portion 2120. The cutter hinge stabilizer 2163 is further configured to inhibit an organ from extending or sliding into, or otherwise interfering with the hinge 2160 and impeding actuation of the cutter assembly 2100 (e.g., preventing the cutter assembly 2100 from fully transitioning from its open configuration to its partially closed configuration, and to its fully closed configuration) when the organ is clamped or in the process of being clamped or when the organ is cut or in the process of being cut. In this manner, interruption of the hinge 2160 during transitioning of the cutter assembly 2100 between configurations can be inhibited by the cutter hinge stabilizer 2163.

The cutter assembly hinge 2160 further includes a cutter hinge stop 2166 configured to limit or set a maximum angle defined between the blade portion 21110 and the blade receptacle portion 2120. Similarly stated, the cutter hinge stop 2166 is configured to limit or prevent relative angular rotation between the blade portion 2110 and the blade receptacle portion 2120 beyond a threshold. As shown in FIGS. 7B and 8B, the cutter hinge stop 2166 includes a projection extending from the blade receptacle portion 2120 that corresponds with and is operably coupled to the blade portion 2110. In use, for example, as the cutter assembly 2100 is moved to the open configuration (e.g., from its partially closed configuration), relative movement of the blade portion 2110 relative to the blade receptacle portion 2120 is limited by contact between the projection of the cutter hinge stop 2166 and the corresponding surface of the blade portion 2110.

The cutter assembly 2100 further includes a cutter assembly latch 2170 configured to, when engaged, releasably retain the cutter assembly 2100 in its fully closed configuration. Said another way, the cutter assembly latch 2170 is configured to, when engaged, limit relative movement between the blade portion 2110 and the blade receptacle portion 2120 (e.g., during storage before or after use of the cutter clamp assembly 2000 in a procedure). The cutter assembly latch 2170 includes a cutter assembly latch projection 2172 extending from the blade portion 2110 and a cutter assembly latch receptacle 2174 defined by the blade receptacle portion 2120 and configured to receive, engage with and be releasably coupled to the cutter assembly latch projection 2172. In use, when the cutter assembly is transitioned to its fully closed configuration (e.g., in response to a manual force provided by a user to the blade portion grip 2116 and the blade receptacle portion grip 2126), the cutter assembly latch projection 2172 is urged into the cutter assembly latch receptacle 2174 beyond a threshold (by way of a snap or interference fit) such that the cutter assembly latch 2170 engages. In this manner, the latch 2170 can self-engage, e.g., without requiring separate manipulation of the latch 2170 by a user. Such self-engagement promotes ease of use by allowing, for example, a single user to perform effectively and safely a cut/clamp procedure with the cutter assembly 2100.

In this embodiment, the cutter assembly latch 2170 can be engaged and disengaged by a user. For example, to disengage the cutter assembly latch 2170, a user can manipulate the cutter assembly latch projection 2172 (e.g., press the latch projection from its biased position) to free or separate the latch projection 2172 from the latch receptacle 2174 such that the cutter assembly 2100 can be transitioned from its fully closed configuration to its partially closed or open configurations. In alternative embodiments, the cutter assembly latch 2170 can be substantially permanently engaged such that the cutter assembly latch 2170 locks the cutter assembly 2100 in its fully closed configuration when the cutter assembly latch 2170 is engaged. In such embodiments, the cutter assembly latch can limit or prevent reuse of the cutter assembly 2100 and potential contamination resulting from such reuse.

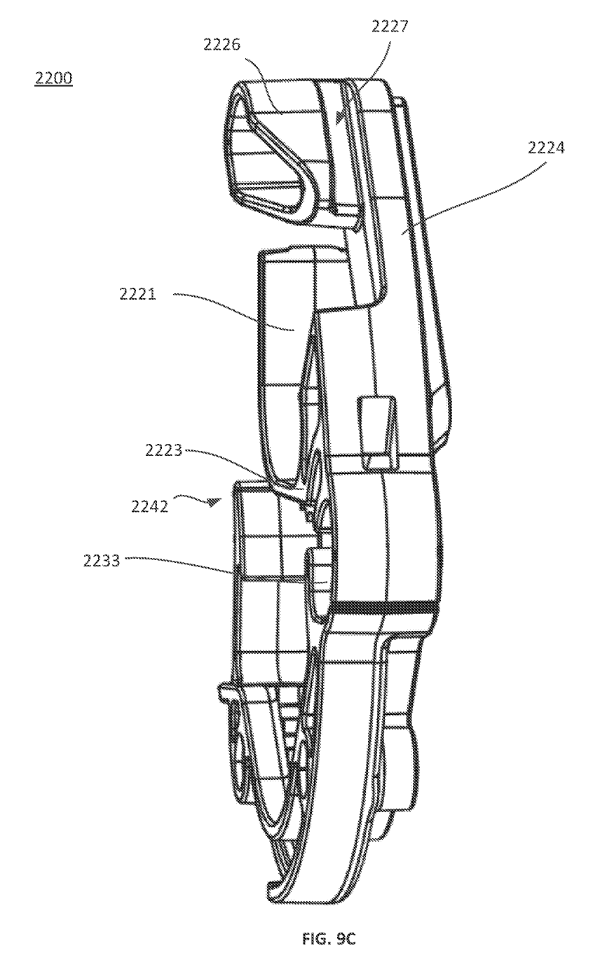

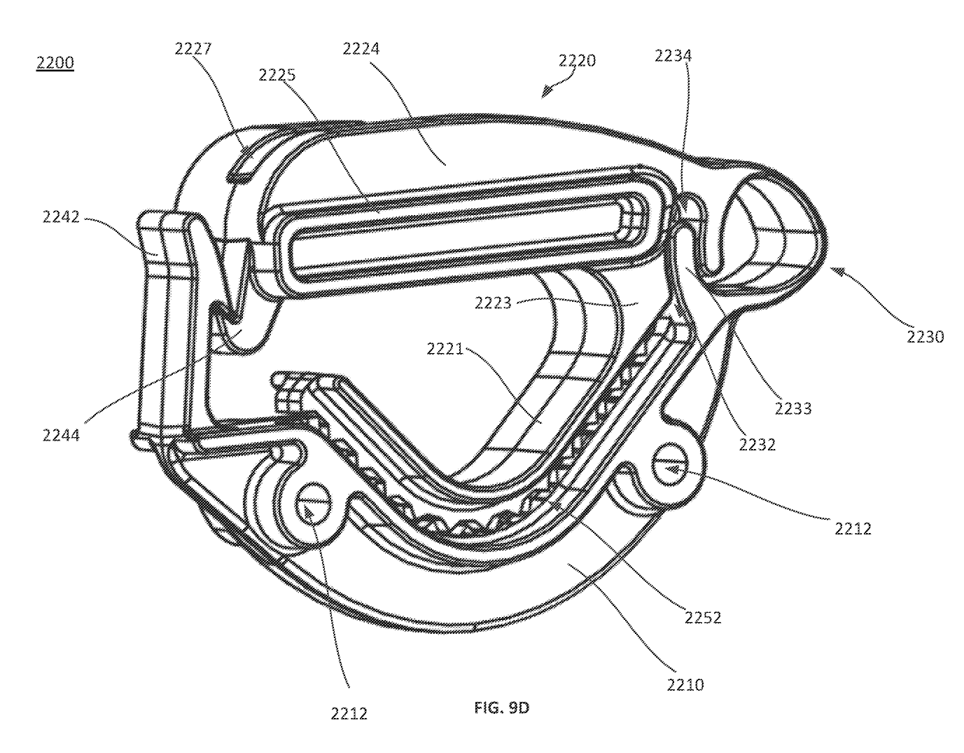

Moreover, as discussed above, the cutter clamp assembly includes a first clamp 2200, as illustrated in FIGS. 9A-C in perspective views, in an open configuration, and in FIG. 9D in perspective view, in a closed configuration. The first clamp 2200 is configured to clamp a portion of an elongate biological organ, and includes a first clamp upper jaw portion 2220 and a first clamp lower jaw portion 2210 rotationally coupled or hinged to the first clamp upper jaw portion 2220 via a first clamp hinge 2230. The first clamp hinge 2230 is configured to promote relative angular movement or otherwise provide a pivot section or point between the first clamp upper jaw portion 2220 and the first clamp lower jaw portion 2210. In this manner, the first clamp can transition between configurations (e.g., open and closed configurations) by the first clamp upper jaw portion 2220 and the first clamp lower jaw portion 2210 rotating relative to each other about the first clamp hinge 2230.

Similar to the discussion with respect to the first clamp 1200, to clamp an organ, the first clamp 2200 can be manipulated from a first, open configuration, to a second, closed configuration.

The first clamp hinge 2230 is configured to be biased to its open configuration. The first clamp hinge 2230 includes a first clamp hinge guard 2232 configured to facilitate clamping (i.e., movement of the first clamp hinge 2230 from its open, biased configuration, to its closed, unbiased configuration) of an organ by providing stabilization or stiffening of the first clamp upper jaw portion 2220, the first clamp lower jaw portion 2210, and the desired dynamics of the first clamp hinge 2230 as the first clamp 2200 transitions between configurations. The first clamp hinge guard 2232 is further configured to limit or prevent the organ from extending or sliding into, or otherwise interfering with the first clamp hinge 2230 (e.g., preventing the first clamp 2230 from fully transitioning to its closed position) when the organ is clamped or in the process of being clamped by the first clamp hinge 2230. As shown, the first clamp hinge guard 2232 includes a first clamp hinge guard projection 2233 and a first clamp hinge guard receptacle 2234 configured to receive or slidably mate with the first clamp hinge guard projection 2233, e.g., when the first clamp 2200 is in its closed configuration. In use, for example, as the first clamp 2200 is manipulated to clamp an organ, i.e., as the first clamp 2200 is transitioned from its open configuration to its closed configuration, the first clamp hinge guard projection 2233 will slide into the first clamp hinge guard receptacle 2234. In this manner, in use, the first clamp hinge guard 2232 provides stiffening or stabilization (e.g., lateral stabilization) to promote proper clamping of an organ while limiting any undesirable counterforces by the organ from contributing to or causing incomplete clamping of the organ. Similarly stated, the first clamp hinge guard 2232 promotes continuous desirable alignment between the first clamp lower jaw 2210 and the first clamp upper jaw portion 2220 during clamping and cutting of the organ.

The first clamp lower jaw 2210 of the first clamp 2200 includes a first clamp organ receiver 2214 configured to receive a portion of an organ to be clamped, and a first clamp lower jaw teeth 2216 configured to grip, hold or otherwise promote retainment of, the portion of the organ to be clamped. In this manner, in use, the first clamp organ receiver 2214 and the first clamp lower jaw teeth 2216 can promote proper positioning and retainment of the organ in preparation for and during clamping of the organ, and during cutting of the organ. The first clamp upper jaw portion 2220 includes a first clamp actuator arm 2224 having stiffening ribs 2225 to provide structural stiffening or support, and a first clamp upper jaw lever 2221 coupled to the actuator arm 2224 via a first clamp upper jaw lever support 2223. The stiffening ribs 2225, in use, for example, provide stabilizing support to the first clamp actuator arm 2224 to resist undesirable movement of the actuator arm 2224 in response to torque generated in part by the interaction of the first clamp 2200 and the cutting timing mechanism 2600, as described in further detail herein. The upper jaw lever 2221, collectively with the first clamp lower jaw 2210, is configured to compress or clamp a portion of the organ when the portion of the organ is disposed in the first clamp organ receiver 2214.

The upper jaw lever 2221 includes first clamp upper jaw teeth 2222 configured to grip, hold or otherwise promote retainment of the portion of the organ to be clamped. Disposed between the first clamp upper jaw lever 2221 and the first clamp actuator arm 2224 is a first clamp upper jaw lever support 2223 (e.g., a fulcrum) configured to provide support about which the upper jaw lever 2221 can pivot or move, e.g., in response to contact with the organ, relative to the first clamp actuator arm 2224, the first clamp hinge 2230 and the first clamp lower jaw 2210.

As discussed in further detail herein, during a clamp and cut procedure, predictable and repeatable timing of the clamping relative to the cutting is important to the effectiveness, safety and overall success of such a procedure. Elongate biological organs, however, vary in size, stiffness and other properties, across various patients. Accordingly, the upper jaw lever 2221, the upper jaw lever support 2223 and the actuator arm 2224 are collectively configured to promote predictable and repeatable clamp and cut timing for a wide range of organs having various properties and characteristics. For example, in use, as the first clamp 2200 is actuated (i.e., transitioned from its open configuration to its closed configuration) and the upper jaw lever 2221 and the lower jaw 2210 come into contact with a portion of the organ, the allowance of relative movement between the first clamp upper jaw lever 2221 and the first clamp actuator arm 2224 allows for suitable clamping of the organ without undesirably affecting the timing of the clamping relative to the subsequent cutting or severing of the organ, as will be discussed in further detail herein with respect to the cutter timer mechanism 2600. Further, such relative movement allows for use of a single-sized clamp for clamping of organs having various sizes, e.g., small diameter organs and large diameter organs, without having to design and manufacture clamps of various sizes to facilitate various sized organs.

The first clamp 2200 further includes a first clamp latch 2240 having a first clamp latch first portion 2242 and a first clamp latch second portion 2244 configured to removably engage with or couple to the first portion 2242. When engaged, the first clamp 2200 is removably locked in its closed configuration. Similarly stated, the first clamp latch 2240, when engaged, is configured to limit relative movement between the first clamp lower jaw 2210 and the first clamp upper jaw portion 2220 (e.g., the first clamp actuator arm 2224 of the upper jaw portion 2220). In this manner, the first clamp 2200 can be secured in its closed configuration during storage prior to use and/or after use. Further, after use, i.e., when a portion of an organ is clamped by the first clamp 2200, the first clamp latch 2240 can secure the organ in its clamped or deformed configuration (e.g., to promote sanitation and limit undesirable contamination of the organ or patient). The first clamp 2200 can be further manipulated to disengage the first clamp latch 2240 and/or transition the first clamp 2200 from its closed configuration to its open configuration. For example, in a procedure involved clamping an umbilical cord, the first clamp 2200 can clamp the umbilical cord to facilitate the cutting of the umbilical cord, and then the first clamp 2200 can be disengaged (moved from its closed configuration to its open configuration) or separated from the umbilical cord such that blood can be withdrawn from the umbilical cord.

In some embodiments, the latch 2240 can self-engage in response to the force applied to transition the first clamp 2200 from its open configuration to its closed configuration. Such self-engagement (e.g., via an interference fit) can provide for desirable locking of the first clamp 2200, as discussed above, without requiring a user of the cutter clamp assembly 2000 from separately manipulating the latch 2240. Such self-engagement can further promote ease of use by allowing, for example, a single user to perform effectively and safely a cut/clamp procedure with the cutter assembly 2100. In alternative embodiments, the latch may require manipulation of the latch by a user to engage the latch.

In yet alternative embodiments, the latch can be substantially permanently engaged such that the latch locks the first clamp in its closed configuration when the latch is engaged or actuated. In such embodiments, the latch can limit or prevent reuse of the clamp and potential complications resulting from removal of the latch from the organ or patient. For example, in procedures in which umbilical cord blood collection is not performed, the latch can remain engaged such that the organ remains clamped by the latch. In such cases, in some instances, the latch can remain clamped about the organ at least until the organ is detached from the patient or when a trained healthcare personnel removes the clamp.

As discussed above and as illustrated, for example, in FIGS. 6A-6C, the first clamp 2200 is removably coupled to the cutter assembly 2100 via the first clamp mount 2300. The first clamp mount 2300 is configured to operably and removably couple the first clamp 2200 to the cutter assembly 2100 such that, as discussed further herein, the first clamp 2200 can be actuated when coupled to the cutter assembly 2100, and decoupled or separated from the cutter assembly 2100 when the first clamp 2200 is in its closed configuration and the cutter assembly 2100 has transitioned from its open configuration its fully closed configuration. In this manner, in use, the first clamp 2200 can separate from the cutter assembly 2100 and maintain clamping of a portion of the organ when the organ has been cut or severed by the cutter assembly 2100.

The first clamp mount 2300 includes a collection of corresponding features of the cutter assembly 2100 and the first clamp 2200. Referring to the cutter assembly 2100, the first clamp mount 2300 includes a first clamp mount cutter portion 2130 of the cutter assembly 2100, defining a first post 2131 and a second post 2132 extending from the blade receptacle portion 2120. Referring to the first clamp 2200, the first clamp lower jaw portion 2210 of the first clamp 2200 includes a first clamp mount clamp portion 2212 configured to releasably mate with the first clamp mount cutter portion 2130 of the cutter assembly 2100. As shown, for example in FIG. 6A, the first clamp mount clamp portion 2212 of the first clamp lower jaw 2210 defines receptacles or sockets configured to receive and slidably and removably mate with the first post 2131 and the second post 2132 of the first clamp mount cutter portion 2130.

As discussed above, in use, a force applied at and to the cutter assembly, e.g., the blade receptacle portion grip 2126 and the blade portion grip 2116, can actuate the cutter clamp assembly 2000 such that relative movement of the blade portion 2110 and the blade receptacle portion 2120 of the cutter assembly 2100 between its open configuration and its partially closed configuration produces relative movement of the first clamp upper jaw portion 2220 and the first clamp lower jaw portion 2210 of the first clamp 2200 between its open configuration and its closed configuration. To facilitate such suitable timing and transfer of forces to cause transitions between such configurations, the cutter clamp assembly 2000 includes a cutting timer mechanism 2600.

The cutting timer mechanism 2600 includes a first clamp cutting timer shoulder 2226 of the first clamp 2200 extending laterally from the first clamp actuator arm 2224. The first clamp cutting timer shoulder 2226 defines a first clamp cutting timer receptacle 2227 configured to slidably couple to or mate with a corresponding first projection 2152 of a cutting timer blade portion 2150 (of the cutting timer mechanism 2600) of the cutter assembly 2100. As shown, for example in FIG. 7, the first projection 2152 of the cutting timer blade portion 2150 extends from the blade 2112 of the cutter assembly 2100, and is configured to slide within or be guided by the first clamp cutting timer receptacle 2227 when the first clamp 2200 is coupled to the cutter assembly 2100 and the cutter assembly 2100 transitions from its open configuration to its partially closed configuration.

Moreover, with the first clamp 2200 in its closed configuration, as the cutter assembly 2100 transitions from its partially closed configuration to its fully closed configuration, the first projection 2152 slides towards and beyond an edge of the first clamp cutting timer receptacle 2227. With the first projection 2152 separated from the first clamp cutting timer receptacle 2227, further relative movement of the cutter assembly 2100 (from its partially closed configurations towards its fully closed configuration) includes the first projection 2152 of the cutting timer blade portion 2150 interfere with and push the first clamp upper jaw portion 2220 such that the first clamp mount cutter portion 2130 of the cutter assembly 2100 is decoupled or separated from the first clamp mount clamp portion 2212, and the first clamp 2200 is decoupled or separated from the cutter assembly 2100.

As discussed herein, the cutter clamp assembly 2000 further includes a second clamp 2400 configured to clamp a portion of an elongate biological organ and removably coupled to the cutter assembly 2100 via the second clamp mount 2500. The second clamp 2400 is the same as and functions the same as the first clamp 2200, but is a mirror image of the first clamp 2200. Similarly, the second clamp mount 2400 is the same as and functions the same as the first clamp mount 2300. For example, the second clamp 2400 includes a second clamp upper jaw portion 2420 and a second clamp lower jaw portion 2410 rotationally coupled or hinged to the second clamp upper jaw portion 2320 via second clamp hinge 2420.

Similar to the discussion with respect to the first clamp 1200 and the first clamp 2200, to clamp an organ, the second clamp 2400 can be manipulated from a first, open configuration, to a second, closed configuration. With the second clamp 2400 disposed about an organ (i.e., the organ is positioned within a second clamp organ receiving zone 2451 defined between the second clamp upper jaw portion 2420 and the second clamp lower jaw portion 2410), the second clamp 2400 can be manipulated (e.g., by a manual force applied by a user to the second clamp upper jaw portion 2420 and/or the second clamp lower jaw portion 2410) to transition the second clamp 2400 from its open configuration to its closed configuration.

The second clamp hinge 2430 is configured to be biased to its open configuration. The second clamp hinge 2430 includes a second clamp hinge guard 2432 configured to facilitate clamping (i.e., movement of the second clamp hinge 2430 from its open, biased configuration, to its closed, unbiased configuration) of an organ by providing stabilization or stiffening of the second clamp upper jaw portion 2420, the second clamp lower jaw portion 2410, and the desired dynamics of the second clamp hinge 2430 as the second clamp 2400 transitions between configurations. The second clamp hinge guard 2432 is further configured to limit or prevent the organ from extending or sliding into, or otherwise interfering with the second clamp hinge 2430 (e.g., preventing the second clamp 2430 from fully transitioning to its closed position) when the organ is clamped or in the process of being clamped by the second clamp hinge 2430. As shown, the second clamp hinge guard 2432 includes a second clamp hinge guard projection 2433 and a second clamp hinge guard receptacle 2434 configured to receive or slidably mate with the second clamp hinge guard projection 2433, e.g., when the second clamp 2400 is in its closed configuration. In use, for example, as the second clamp 2400 is manipulated to clamp an organ, i.e., as the second clamp 2400 is transitioned from its open configuration to its closed configuration, the second clamp hinge guard projection 2433 will slide into the second clamp hinge guard receptacle 2434. In this manner, in use, the second clamp hinge guard 2432 provides stiffing or stabilization (e.g., lateral stabilization) to promote proper clamping of an organ while limiting any undesirable counterforces by the organ from contributing to or causing incomplete clamping of the organ. Similarly stated, the second clamp hinge guard 2432 promotes continuous desirable alignment between the second clamp lower jaw 2410 and the second clamp upper jaw portion 2420 during clamping and cutting of the organ.

The second clamp lower jaw 2410 of the second clamp 2400 includes a second clamp organ receiver 2414 configured to receive a portion of an organ to be clamped, and a second clamp lower jaw teeth 2416 configured to grip, hold or otherwise promote retainment of the portion of the organ to be clamped. In this manner, in use, the second clamp organ receiver 2414 and the second clamp lower jaw teeth 2416 can promote proper positioning and retainment of the organ in preparation for and during clamping of the organ, and during cutting of the organ.

The second clamp upper jaw portion 2420 includes a second clamp actuator arm 2424 having stiffening ribs 2425 to provide structural stiffening or support, and a second clamp upper jaw lever 2421 coupled to the actuator arm 2424 via a second clamp upper jaw lever support 2423. The stiffening ribs 2425, in use, for example, provide stabilizing support to the second clamp actuator arm 2424 to resist undesirable movement of the actuator arm 2424 in response to torque generated in part by the interaction of the second clamp 2400 and the cutting timing mechanism 2600, as described in further detail herein. The upper jaw lever 2421, collectively with the second clamp lower jaw 2410, is configured to compress or clamp a portion of the organ when the portion of the organ is disposed in the second clamp organ receiver 2414.

The upper jaw lever 2421 includes second clamp upper jaw teeth 2422 configured to grip, hold or otherwise promote retainment of the portion of the organ to be clamped. Disposed between the second clamp upper jaw lever 2421 and the second clamp actuator arm 2424 is a second clamp upper jaw lever support 2423 (e.g., a fulcrum) configured to provide support about which the upper jaw lever 2421 can pivot or move, e.g., in response to contact with the organ, relative to the second clamp actuator arm 2424, the second clamp hinge 2430 and the second clamp lower jaw 2410.

As discussed in further detail herein, during a clamp and cut procedure, predictable and repeatable timing of the clamping relative to the cutting is important to the effectiveness, safety and overall success of such a procedure. Elongate biological organs, however, vary in size, stiffness and other properties, across various patients. Accordingly, the upper jaw lever 2421, the upper jaw lever support 2423 and the actuator arm 2424 are collectively configured to promote predictable and repeatable clamp and cut timing for a wide range of organs having various properties and characteristics. For example, in use, as the second clamp 2400 is actuated (i.e., transitioned from its open configuration to its closed configuration) and the upper jaw lever 2421 and the lower jaw 2410 come into contact with a portion of the organ, the allowance of relative movement between the second clamp upper jaw lever 2421 and the second clamp actuator arm 2424 allows for suitable clamping of the organ without undesirably affecting the timing of the clamping relative to the subsequent cutting or severing of the organ, as will be discussed in further detail herein with respect to the cutter timer mechanism 2600. Further, such relative movement allows for use of a single-sized clamp for clamping of organs having various sizes, e.g., small diameter organs and large diameter organs, without having to design and manufacture clamps of various sizes to facilitate various sized organs.

The second clamp 2400 further includes a second clamp latch 2440 having a second clamp latch second portion 2442 and a second clamp latch second portion 2444 configured to removably engage with or couple to the second portion 2442. When engaged, the second clamp 2400 is removably locked in its closed configuration. Similarly stated, the second clamp latch 2440, when engaged, is configured to limit relative movement between the second clamp lower jaw 2410 and the second clamp upper jaw portion 2420 (e.g., the second clamp actuator arm 2424 of the upper jaw portion 2420). In this manner, the second clamp 2400 can be secured in its closed configuration during storage prior to use and/or after use. Further, after use, i.e., when a portion of an organ is clamped by the second clamp 2400, the second clamp latch 2440 can secure the organ in its clamped or deformed configuration (e.g., to promote sanitation and limit undesirable contamination of the organ or patient). The second clamp 2400 can be further manipulated to disengage the second clamp latch 2440 and/or transition the second clamp 2400 from its closed configuration to its open configuration. For example, in a procedure involved clamping an umbilical cord, the second clamp 2400 can clamp the umbilical cord to facilitate the cutting of the umbilical cord, and then the second clamp 2400 can be disengaged (moved from its closed configuration to its open configuration) or separated from the umbilical cord such that blood can be withdrawn from the umbilical cord.

In some embodiments, the latch 2440 can self-engage in response to the force applied to transition the second clamp 2400 from its open configuration to its closed configuration. Such self-engagement (e.g., via an interference fit) can provide for desirable locking of the second clamp 2400, as discussed above, without requiring a user of the cutter clamp assembly 2000 from separately manipulating the latch 2440. Such self-engagement can further promote ease of use by allowing, for example, a single user to perform effectively and safely a cut/clamp procedure with the cutter assembly 2100. In alternative embodiments, the latch may require manipulation of the latch by a user to engage the latch.

In yet alternative embodiments, the latch can be substantially permanently engaged such that the latch locks the second clamp in its closed configuration when the latch is engaged or actuated. In such embodiments, the latch can limit or prevent reuse of the clamp and potential complications resulting from removal of the latch from the organ or patient. For example, in procedures in which umbilical cord blood collection is not performed, the latch can remain engaged such that the organ remains clamped by the latch. In such cases, in some instances, the latch can remain clamped about the organ at least until the organ is detached from the patient or when a trained healthcare personnel removes the clamp.

As discussed above and as illustrated, for example, in FIGS. 6A-6C, the second clamp 2400 is removably coupled to the cutter assembly 2100 via the second clamp mount 2300. The second clamp mount 2300 is configured to operably and removably couple the second clamp 2400 to the cutter assembly 2100 such that, as discussed further herein, the second clamp 2400 can be actuated when coupled to the cutter assembly 2100, and decoupled or separated from the cutter assembly 2100 when the second clamp 2400 is in its closed configuration and the cutter assembly 2100 has transitioned from its open configuration its fully closed configuration. In this manner, in use, the second clamp 2400 can separate from the cutter assembly 2100 and maintain clamping of a portion of the organ when the organ has been cut or severed by the cutter assembly 2100.

The second clamp mount 2300 includes a collection of corresponding features of the cutter assembly 2100 and the second clamp 2400. Referring to the cutter assembly 2100, the second clamp mount 2300 includes a second clamp mount cutter portion 2140 of the cutter assembly 2100, defining a first post 2141 and a second post 2142 extending from the blade receptacle portion 2120. Referring to the second clamp 2400, the second clamp lower jaw portion 2410 of the second clamp 2400 includes a second clamp mount clamp portion 2412 configured to releasably mate with the second clamp mount cutter portion 2140 of the cutter assembly 2100. As shown, for example in FIG. 6A, the second clamp mount clamp portion 2412 of the second clamp lower jaw 2410 defines receptacles or sockets configured to receive and slidably and removably mate with the second post 2131 and the second post 2132 of the second clamp mount cutter portion 2140.

As discussed above, in use, a force applied at and to the cutter assembly, e.g., the blade receptacle portion grip 2126 and the blade portion grip 2116, can actuate the cutter clamp assembly 2000 such that relative movement of the blade portion 2110 and the blade receptacle portion 2120 of the cutter assembly 2100 between its open configuration and its partially closed configuration produces relative movement of the second clamp upper jaw portion 2420 and the second clamp lower jaw portion 2410 of the second clamp 2400 between its open configuration and its closed configuration. To facilitate such suitable timing and transfer of forces to cause transitions between such configurations, the cutter clamp assembly 2000 includes a cutting timer mechanism 2600.

The cutting timer mechanism 2600 includes a second clamp cutting timer shoulder 2426 of the second clamp 2400 extending laterally from the second clamp actuator arm 2424. The second clamp cutting timer shoulder 2426 defines a second clamp cutting timer receptacle 2427 configured to slidably couple to or mate with a corresponding second projection 2154 of a cutting timer blade portion 2150 (of the cutting timer mechanism 2600) of the cutter assembly 2100. As shown, for example in FIG. 7, the second projection 2154 of the cutting timer blade portion 2150 extends from the blade 2112 of the cutter assembly 2100, and is configured to slide within or be guided by the second clamp cutting timer receptacle 2427 when the second clamp 2400 is coupled to the cutter assembly 2100 and the cutter assembly 2100 transitions from its open configuration to its partially closed configuration.

Moreover, with the second clamp 2400 in its closed configuration, as the cutter assembly 2100 transitions from its partially closed configuration to its fully closed configuration, the second projection 2154 slides towards and beyond an edge of the second clamp cutting timer receptacle 2427. With the second projection 2154 separated from the second clamp cutting timer receptacle 2427, further relative movement of the cutter assembly 2100 (from its partially closed configurations towards its fully closed configuration) includes the second projection 2154 of the cutting timer blade portion 2150 interfere with and push the second clamp upper jaw portion 2420 such that the second clamp mount cutter portion 2140 of the cutter assembly 2100 is decoupled or separated from the second clamp mount clamp portion 2412, and the second clamp 2400 is decoupled or separated from the cutter assembly 2100.

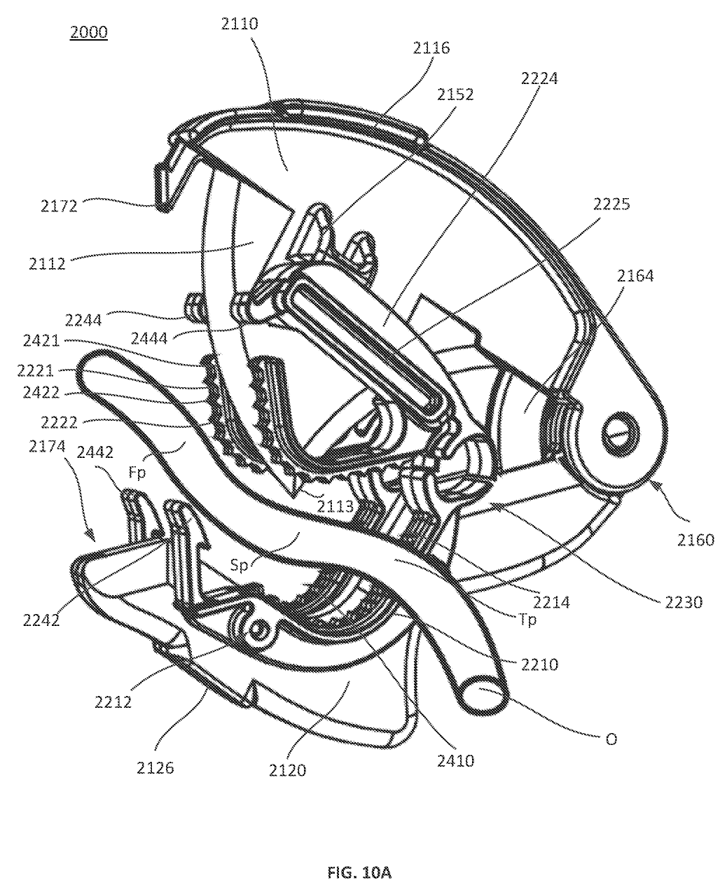

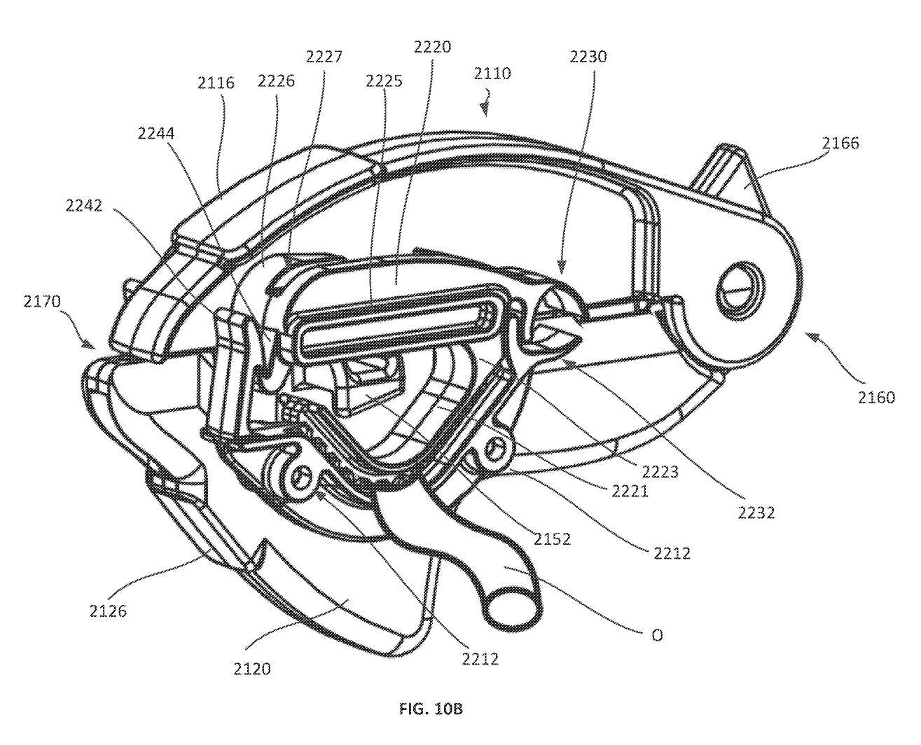

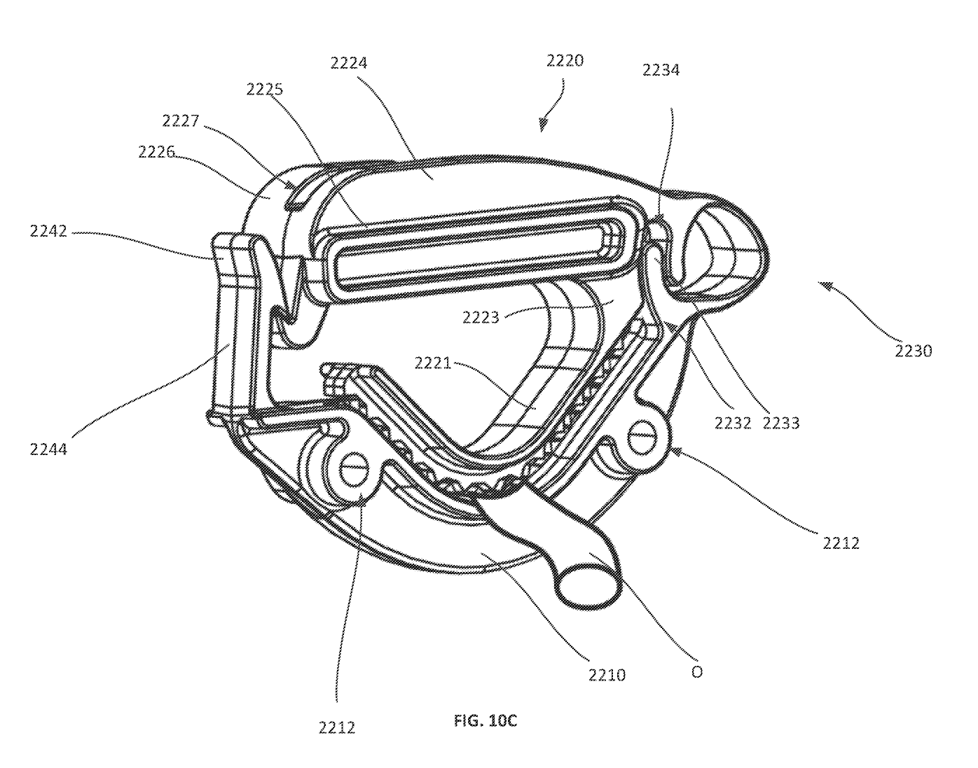

In use, as illustrated in FIGS. 10A-10C, during an umbilical cord procedure, for example, with the cutter assembly 2100 coupled to the first clamp 2200 via the first clamp mount 2300 and the cutter assembly 2100 coupled to the second clamp 2400 via the second clamp mount 2500, the umbilical cord can be disposed across the cutter clamp assembly 2000 in preparation for clamping and cutting of the umbilical cord (see e.g., FIG. 10A). More specifically, a first portion “Fp” of the umbilical cord can be disposed across the first clamp organ receiver 2214 and within the first clamp organ receiving zone 2251. A second portion “Sp” of the umbilical cord can be disposed across the blade receptacle portion organ receiver 2124 and within the cutting zone 2180. A third portion “Tp” of the umbilical cord can be disposed across the second clamp organ receiver 2414 and within the second clamp organ receiving zone 2451. In this manner, as illustrated in FIG. 10A, the second portion of the umbilical cord (i.e., the portion of the umbilical cord to be cut) is disposed between the first and third portions of the umbilical cord (i.e., the portions of the umbilical cord to be clamped).

Due in part to the symmetry on either side of the blade 2112, a user can approach the umbilical cord from various sides and angles. In some instances, for example, the first portion of the umbilical cord can be on the placenta-side of the umbilical cord while the third portion of the umbilical cord is on the newborn-side of the umbilical cord. In other instances, for example, the first portion of the umbilical cord can be on the newborn-side and the third portion of the umbilical cord can be on the placenta side. Such a configuration promotes repeatable approaches regardless of the particular user, and similarly limits potential for mistakes (e.g., by an untrained user).

With the umbilical cord suitably positioned relative to the cutter clamp assembly 2000, pressure (e.g., manual pressure from a user) can be applied to the blade portion 2110 of the cutter assembly 2100 and the blade receptacle portion 2120 of the cutter assembly 2100 to transition the cutter assembly 2100 from its open configuration to its partially closed configuration and thus to transition (1) the first clamp 2200 from its open configuration to its closed configuration and to clamp the first portion of the umbilical cord between the first clamp lower jaw 2210 and the first clamp upper jaw lever 2221, and (2) the second clamp 2400 from its open configuration to its closed configuration and to clamp the third portion of the umbilical cord between the second clamp lower jaw 2410 and the second clamp upper jaw lever 2421.

As discussed herein, the pressure applied to transition the cutter assembly 2100 to its partially closed configuration causes the first projection 2152 of the cutting timer blade portion 2150 to slide along the first clamp cutting timer receptacle 2227 of the first clamp cutting timer shoulder 2226 such that the pressure is transferred from the first projection to the first clamp 2200 to urge the first clamp 2200 to its closed configuration. Further, the first clamp latch 2240 engages when the first clamp 2200 is in its closed configuration and the first portion of the organ is clamped by the first clamp 2200.

Similarly, the pressure applied to transition the cutter assembly 2100 to its partially closed configuration causes the second projection 2252 of the cutting timer blade portion 2150 to slide along the second clamp cutting timer receptacle 2427 of the second clamp cutting timer shoulder 2426 such that the pressure is transferred from the second projection to the second clamp 2400 to urge the second clamp 2400 to its closed configuration. Further, the second clamp latch 2440 engages when the second clamp 2400 is in its closed configuration and the third portion of the organ is clamped by the second clamp 2400.

In some instances, with the cutter assembly 2100 in its partially closed configuration, the blade tip 2113 can pierce and at least partially cut the second portion of the organ such that the blade portion 2112 is in communication with contents (e.g., blood) of the organ. With the first clamp 2200 and the second clamp 2400 in close proximity to the blade 2110 (i.e., on either side of the blade 2110), content splatter in response to the cutting and clamping is limited or at least partially contained in the cutter clamp assembly 2000, thereby promoting sanitation of the area surrounding the procedure. Said another way, the first clamp 2200 and the second clamp 2400, in some instances, can intercept or block content from splattering from the cutter clamp assembly 2000.

Further, with the first clamp 2200 and the second clamp 2400 and their closed configurations, further pressure applied to transition the cutter assembly 2100 towards its fully closed configuration causes the first clamp 2200 and the second clamp 2400 to separate from the cutter assembly 2100, with the first clamp 2200 retained on the first portion of the organ and the second clamp 2400 retained on the third portion of the organ.

Further pressure (e.g., manual pressure by a user) can be applied to the blade portion 2110 of the cutter assembly 2100 and the blade receptacle portion 2120 of the cutter assembly 2100 to transition the cutter assembly 2100 from its partially closed configuration to its fully closed configuration (see e.g., FIG. 10B), and to cut or sever the second portion of the organ. Upon the further pressure, the cutter assembly latch 2170 engages when the cutter assembly 2100 is in its fully closed configuration such that further relative movement between the blade portion 2110 and the blade receptacle portion 2120 is limited.

As illustrated in FIG. 10C, upon severance of the umbilical cord, the first clamp 2200 can be separated from the cutter assembly 2100 while remaining clamped to a portion of the umbilical cord.

In an alternative embodiment, a cutter clamp assembly can be configured similar to or the same as the cutter clamp assembly 2000, except that a cutter hinge of the cutter assembly includes a ratchet mechanism configured to allow movement of the cutter assembly from its open configuration to its partially closed configuration, and further to its fully closed configuration, but to limit movement in a reversed direction, i.e., from the fully closed configuration to the partially closed configuration, and from the partially closed configuration to the open configuration. In this manner, when a cutter clamp assembly having a ratchet mechanism is used to clamp and cut an elongate biological organ, the ratchet mechanism can limit or prevent opening or removal of the cutter assembly prior to complete actuation or complete or suitable severing of the organ. In some embodiments, the ratchet mechanism can substantially permanently secure the cutter assembly in its closed configuration.

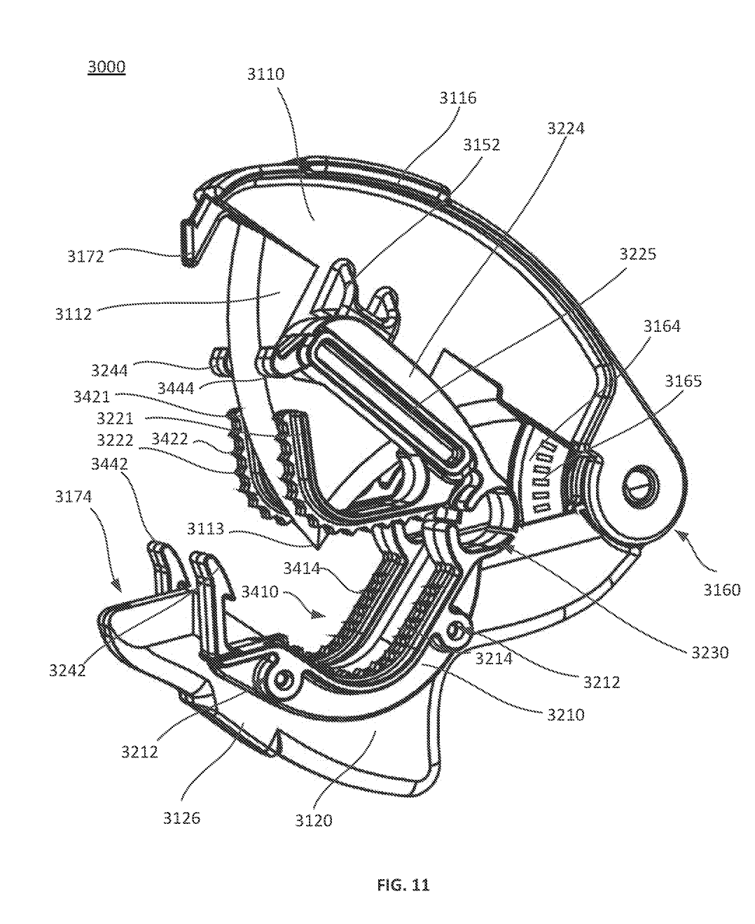

FIG. 11 illustrates a cutter clamp assembly 3000 having a cutter hinge ratchet 3165. The cutter clamp assembly 3000 can be constructed and function similar to any of the cutter clamp assemblies described herein, e.g., the cutter clamp assembly 2000. Thus, some details regarding the cutter clamp assembly 3000 are not described below. It should be understood that for features and functions not specifically discussed, those features and functions can be the same as or similar to any of the cutter clamp assemblies described herein.

As shown in FIG. 11, the cutter clamp assembly 3000 includes a cutter hinge ratchet 3165. The cutter hinge ratchet 3165 is configured to allow movement of the cutter hinge stabilizer projection 3164 (projecting from the blade portion 3110) relative to the cutter hinge stabilizer receptacle (or guide) 3167 when the projection 3164 is disposed within the stabilizer receptacle 3167 and the cutter assembly 3000 is moving from an open configuration to a partially closed configuration or from a partially closed configuration to a fully closed configuration. Further, the cutter hinge ratchet 3165 is configured to limit or prevent movement of the cutter hinge stabilizer projection 3164 from the cutter hinge stabilizer receptacle 3167 when the projection 3164 is disposed within the stabilizing receptacle 3167. Similarly stated, the cutter hinge ratchet 3165 limits or prevents the cutter assembly 3100 from transitioning from a closed configuration to a partially closed configuration or from the partially closed configuration to an open configuration. In this manner, when the cutter clamp assembly 3000 is used to clamp and cut an elongate biological organ, the ratchet mechanism can limit or prevent opening or removal of the cutter assembly 3000 prior to complete actuation or complete or suitable severing of the organ. Further, the cutter hinge ratchet 3165 can limit or prevent reuse of the cutter assembly (and the cutter clamp assembly), thereby limiting insanitation and infections due to such reuse.

In an alternative embodiment, a cutter clamp assembly can be configured similar to or the same as the cutter clamp assembly 3000, except that the cutter assembly excludes a cutter assembly latch. In such embodiments, the cutter hinge ratchet can be configured to lock the cutter assembly in its fully closed configuration (e.g., after the cutter assembly is used in a procedure) such that the cutter assembly cannot transition from its fully closed configuration (e.g., from its fully closed configuration to its partially closed configuration or its open configuration).

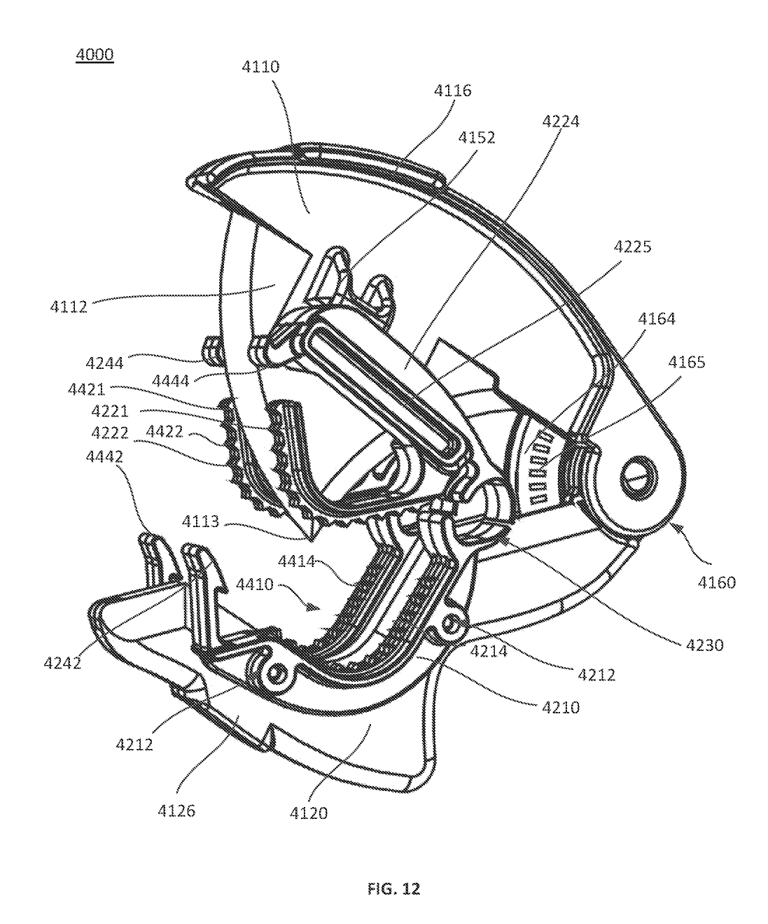

FIG. 12 illustrates a cutter clamp assembly 4000 having a cutter hinge ratchet 3165 and excluding a cutter assembly latch 2170. The cutter clamp assembly 4000 can be constructed and function similar to any of the cutter clamp assemblies described herein, e.g., the cutter clamp assembly 1000, 2000, and/or 3000. Thus, some details regarding the cutter clamp assembly 4000 are not described below. It should be understood that for features and functions not specifically discussed, those features and functions can be the same as or similar to any of the cutter clamp assemblies described herein.

As shown in FIG. 12, the cutter clamp assembly 4000 includes a cutter hinge ratchet 4165. The cutter hinge ratchet 4165 is configured to allow movement of the cutter hinge stabilizer projection 4165 (projecting from the blade portion 4110) relative to the cutter hinge stabilizer receptacle (or guide) 4167 when the projection 4164 is disposed within the stabilizer receptacle 4167 and the cutter assembly 4000 is moving from an open configuration to a partially closed configuration or from a partially closed configuration to a fully closed configuration. Further, the cutter hinge ratchet 4165 is configured to limit or prevent movement of the cutter hinge stabilizer projection 4164 from the cutter hinge stabilizer receptacle 4167 when the projection 3164 is disposed within the stabilizing receptacle 4167. Similarly stated, the cutter hinge ratchet 4165 limits or prevents the cutter assembly 4100 from transitioning from a closed configuration to a partially closed configuration or from the partially closed configuration to an open configuration. In this manner, when the cutter clamp assembly 4000 is used to clamp and cut an elongate biological organ, the ratchet mechanism can limit or prevent opening or removal of the cutter assembly 4000 prior to complete actuation or complete or suitable severing of the organ. Further, the cutter hinge ratchet 4165 can limit or prevent reuse of the cutter assembly (and the cutter clamp assembly), thereby limiting insanitation and infections due to such reuse.

In alternative embodiments, cutter clamp assemblies can be constructed and function similar to or the same as any of the cutter clamp assemblies described above, but can include a blade portion grip and/or a blade receptacle portion grip having a greater width over a greater extent of each side of the cutter assembly, and/or can define finger indentations for improved comfort and more secured grip, thereby promoting effective, repeatable (i.e., consistent use across various users), and easy actuation of the cutter clamp assembly by a user. In some embodiments, a blade portion grip and/or a blade receptacle portion grip can include one or more eye loops (e.g., allowing a user to slide his/her finger therethrough) to promote more stability, grip, and a better transfer of force from the user to the cutter clamp assembly. In some embodiments, a blade portion grip and/or a blade receptacle portion grip can extend beyond one or more edges of the blade portion and/or the blade receptacle portion, thereby providing more grip surface area for a user, and/or providing improved torque (e.g., due to a greater distance of the applied force by the user from the cutter assembly hinge) when a user applies a manual force to the cutter clamp assembly.

Optionally, an antibacterial agent, such as Chlorhexidine, can be packaged with a cutter-clamp assembly (e.g., any of the cutter-clamp assemblies described herein) so that the antibacterial agent can be applied to the organ (e.g., the umbilical cord) stump after delivery. As another option, the packaged antibacterial agent, such as Chlorhexidine, may be laid and/or stabilized across the cutting zone of the cutter assembly so that the package is opened and automatically dispersed on the organ during clamping and cutting of the organ. Said another way, actuation of one or more clamps, or actuation of the cutter assembly, can cause the antibacterial agent (e.g., by piercing a package containing the antibacterial agent) to be released at or near the cutting or clamping zones, thereby limiting complications resulting of insanitation or infections.

While various embodiments have been described above, it should be understood that they have been presented in a way of example only, and not limitation. Where schematics and/or embodiments described above indicate certain components arranged in certain orientations or positions, the arrangement of components may be modified. While the embodiments have been particularly shown and described, it will be understood that various changes in form and details may be made.

Although various embodiments have been described as having particular features and/or combinations of components, other embodiments are possible having a combination of any features and/or components from any of the embodiments discussed above.

1. Apparatus comprising:
a clamp having a lower jaw portion and an upper jaw portion coupled for relative movement between a first, open configuration and a second, closed configuration, the clamp defining between the upper jaw portion and the lower jaw portion an organ receiving zone in the open configuration of the clamp and a clamped zone between the upper jaw portion and the lower jaw portion in the closed configuration of the clamp, the clamp being configured to receive a first portion of an elongate compressible biological organ in the organ receiving zone and to compress the received first portion of the organ between the upper jaw portion and the lower jaw portion into the clamped zone;
a cutter assembly having a first portion and a second portion coupled for relative movement between a first, open configuration, a second, partially closed configuration, and a third, fully closed configuration, one of the first portion and the second portion including a blade projecting toward the other of the first portion and the second portion, the cutter assembly defining a cutting zone between the first portion and the second portion, the blade being at least partially clear of the cutting zone when the cutter assembly is disposed in the partially closed configuration and the blade being disposed completely across the cutting zone when the cutter assembly is disposed in the fully closed configuration,
the cutter assembly releasably coupled to the clamp by a mounting connection between the lower jaw portion of the clamp and the second portion of the cutter assembly, with the clamped zone of the clamp disposed laterally adjacent to the cutting zone of the cutter assembly, such that a second portion of the elongate biological organ can be received in the cutting zone when the first portion of the organ is disposed in the clamped zone;
a cutting timer mechanism having a first timer portion disposed on the upper jaw portion of the clamp and a second timer portion disposed on the first portion of the cutter assembly, the first timer portion engageable with the second timer portion such that relative movement of the first portion and the second portion of the cutter assembly between the open configuration and the partially closed configuration of the cutter assembly produces relative movement of the upper jaw portion and the lower jaw portion between the open configuration and the closed configuration of the clamp, the cutting timer mechanism configured to permit further relative movement of the first portion and the second portion of the cutter assembly between the partially closed configuration and the fully closed configuration of the cutter assembly without further relative movement of the upper jaw portion and the lower jaw portion of the clamp.

2. The apparatus of claim 1,
wherein the cutter assembly has a first lateral side and a second lateral side, opposite to the first lateral side, the first clamp being releasably coupled to the cutter assembly on the first lateral side of the cutter assembly,
wherein the clamp is a first clamp, the organ receiving zone is a first organ receiving zone, the clamped zone is a first clamped zone, and the mounting connection is a first mounting connection;
further comprising a second clamp, the second clamp having a lower jaw portion, an upper jaw portion and a clamp hinge coupling the lower jaw portion and the upper jaw portion for relative movement between a first, open configuration and a second, closed configuration, the second clamp defining between the upper jaw portion and the lower jaw portion a second organ receiving zone in the open configuration of the clamp and a second clamped zone between the upper jaw portion and the lower jaw portion in the closed configuration of the second clamp, the second clamp being configured to receive a third portion of the elongate compressible biological organ in the organ receiving zone and to compress the received third portion of the organ between the upper jaw portion and the lower jaw portion into the second clamped zone;
the cutter assembly releasably coupled to the second clamp by a second mounting connection between the lower jaw portion of the first clamp and the second portion of the cutter assembly, with the second clamped zone of the second clamp disposed laterally adjacent to the cutting zone of the cutter assembly, on the opposite lateral side cutting zone from the first clamped zone, such that a the third portion of the elongate biological organ can be received in the second clamped zone when the second portion of the organ is received in the cutting zone and the first portion of the organ is disposed in the first clamped zone.

3. The apparatus of claim 2, wherein the cutting timer mechanism has a third timer portion disposed on the upper jaw portion of the second clamp and a fourth timer portion disposed on the first portion of the cutter assembly, the third timer portion engageable with the fourth timer portion such that relative movement of the first portion and the second portion of the cutter assembly between the open configuration and the partially closed configuration of the cutter assembly produces relative movement of the upper jaw portion and the lower jaw portion of the second clamp between the open configuration and the closed configuration of the second clamp, the cutting timer mechanism configured to permit further relative movement of the first portion and the second portion of the cutter assembly between the partially closed configuration and the fully closed configuration of the cutter assembly without further relative movement of the upper jaw portion and the lower jaw portion of the second clamp.

4. The apparatus of claim 1, wherein the blade is disposed on the first portion of the cutter assembly, and the second portion of the cutter assembly includes a blade receptacle, at least a tip portion of the blade being disposed in the blade receptacle when the cutter assembly is disposed in the fully closed position.

5. The apparatus of claim 1, wherein the blade is disposed on the first portion of the cutter assembly, and the first portion of the cutter assembly is monolithically formed of a polymer material.

6. The apparatus of claim 1, wherein the upper jaw portion of the clamp includes an actuator and a lever coupled to the actuator via a support portion,
the first timer portion extending from the actuator,
the lever and the actuator being coupled via the support portion for relative movement, both the organ receiving zone and the clamped zone defined between the lever and the lower jaw portion, the clamp being configured to receive the first portion of the elongate compressible biological organ to compress the received first portion of the organ between the level and the lower jaw portion into the clamped zone.

7. The apparatus of claim 1, wherein the mounting connection includes (1) a socket defined by the lower jaw portion of the clamp, and (2) a post extending from the second portion of the cutter assembly, the socket configured to receive the post when the clamp is coupled to the cutter assembly.

8. The apparatus of claim 1, wherein the first timer portion includes a projection extending laterally from the upper jaw portion of the clamp, the second timer portion including a shoulder extending from the first portion of the cutter assembly, the shoulder defining a receptacle configured to receive at least a portion of the projection.

9. The apparatus of claim 1, wherein the clamp includes a clamp hinge coupling the lower jaw portion and the upper jaw portion for pivotal relative movement between the open configuration and the closed configuration.

10. The apparatus of claim 1, wherein the cutter assembly includes a cutter hinge coupling the first portion and the second portion of the cutter assembly for relative pivotal movement between the open configuration and the fully closed configuration.

11. The apparatus of claim 10, wherein the cutter hinge includes a ratchet mechanism that permits relative pivotal movement from the open configuration towards the fully closed configuration, but inhibits relative pivotal movement in the opposite direction.

12. The apparatus of claim 1, wherein the clamp includes a latch configured to retain the clamp in the closed configuration.

13. The apparatus of claim 12, wherein the latch is releasable.

14. The apparatus of claim 1, wherein the cutter assembly includes a latch configured to retain the cutter assembly in the fully closed configuration.

15. The apparatus of claim 14, wherein the latch is releasable.

16. The apparatus of claim 1, wherein the cutter assembly includes a ratchet configured to retain the cutter assembly in the fully closed configuration.

17. The apparatus of claim 1, wherein the clamp includes an identification device.

18. The apparatus of claim 2, wherein each of the first clamp and the second clamp bears a matching identification or visual indicium.

19. The apparatus of claim 1, wherein each of the blade portion and the blade receptacle portion of the cutter assembly includes a grip portion to which a user can comfortably apply sufficient manual pressure to urge the cutter assembly from the open configuration to the fully closed configuration.

20. The apparatus of claim 1, wherein the cutter assembly includes a hinge stop mechanism configured to limit relative movement between the first portion and the second portion of the cutter assembly beyond a threshold when the cutter assembly is in the open configuration.

21. A method comprising:
disposing about an elongate biological organ a cutter clamp assembly,
the cutter clamp assembly having a clamp with a lower jaw portion and an upper jaw portion coupled for relative movement between an open configuration defining an organ receiving zone therebetween and a closed configuration defining a clamped zone therebetween,
the cutter clamp assembly further including a cutter assembly having a blade portion and a blade receptacle portion coupled for relative movement between a first, open configuration, a second, partially closed configuration, and a third, fully closed configuration, the blade portion including a blade projecting toward the receptacle portion, the cutter assembly defining a cutting zone between the blade portion and the blade receptacle portion, the blade being at least partially clear of the cutting zone when the cutter assembly is disposed in the partially closed configuration and the blade being disposed completely across the cutting zone when the cutter assembly is disposed in the fully closed configuration,
the cutter assembly releasably coupled to the clamp with the clamped zone of the clamp disposed laterally adjacent to the cutting zone of the cutter assembly,
the cutter clamp assembly further including a cutting timer mechanism configured such that relative movement of the blade portion and the blade receptacle portion between the open configuration and the partially closed configuration of the cutter assembly produces relative movement of the upper jaw portion and the lower jaw portion between the open configuration and the closed configuration of the clamp, the cutting timer mechanism configured to permit further relative movement of the blade portion and the blade receptacle portion between the partially closed configuration and the fully closed configuration of the cutter assembly without further relative movement of the upper jaw portion and the lower jaw portion of the clamp,
the disposing about the organ including disposing a first portion of the organ in the organ receiving zone of the clamp and disposing a second portion of the organ in the cutting zone of the cutter assembly;
applying manual pressure to the blade portion and the blade receptacle portion of the cutter assembly to urge the cutter assembly from the open configuration to the partially closed configuration and thus to urge the clamp from the open configuration to the closed configuration and to clamp the first portion of the elongate biological organ;
applying further manual pressure to the blade portion and the blade receptacle portion to urge the cutter assembly from the partially closed configuration to the fully closed configuration and to cut the second portion of the organ; and
separating the clamp from the cutter assembly, with the clamp retained on the second portion of the organ.

22. The method of claim 21, wherein the elongate biological organ is an umbilical cord.

23. The method of claim 22, wherein the second portion of the umbilical cord is attached to a placenta, and further comprising:
releasing the clamp from the second portion of the umbilical cord; and
withdrawing blood from the umbilical cord.

24. The method of claim 21, where in the applying manual pressure and the applying further manual pressure is performed in a continuous manual movement.

25. The method of claim 21, wherein
the clamp is a first clamp, the organ receiving zone is a first organ receiving zone, and the clamped zone is a first clamped zone,
the cutter clamp assembly includes a second clamp with a lower jaw portion and an upper jaw portion coupled for relative movement between an open configuration defining an second organ receiving zone therebetween and a closed configuration defining a second clamped zone therebetween, the cutter assembly releasably coupled to the second clamp with the second clamped zone of the second clamp disposed laterally adjacent to the cutting zone of the cutter assembly, on the opposite side of the cutting zone from the first clamped zone,
the disposing about the organ includes disposing a third portion of the organ in the second organ receiving zone,
the applying manual pressure urges the second clamp to the closed configuration of the second clamp to clamp the third portion of the organ, and
further comprising separating the second clamp from the cutter assembly, with the second clamp retained on the third portion of the organ.
